(19) United States
(12) Patent Application Publication
(10) Pub. No.: US 2022/0061743 A1
(43) Pub. Date: Mar. 3, 2022

(54) DETECTION OF SEIZURE AND STROKE

(71) Applicant: Medtronic, Inc., Minneapolis, MN (US)

(72) Inventors: Eric M. Christensen, Gilbert, AZ (US); Philip E. Tracton, Porter Ranch, CA (US); Walton W. Baxter, III, San Clemente, CA (US); Eric J. Panken, Edina, MN (US); John Wainwright, Foothill Ranch, CA (US); Randal C. Schulhauser, Phoenix, AZ (US); Jonathon E. Giftakis, Maple Grove, MN (US); Nathalie Virag, Cottens (CH); Avram Scheiner, Vadnais Heights, MN (US); Ekaterina Ippolito, Shoreview, MN (US)

(21) Appl. No.: 17/459,466

(22) Filed: Aug. 27, 2021

Related U.S. Application Data
(60) Provisional application No. 63/071,828, filed on Aug. 28, 2020.

(51) Int. Cl. A61B 5/00; A61B 5/31; A61B 5/374; A61B 5/28; A61B 5/293; G16H 50/20; G16H 50/30

(52) U.S. Cl. CPC A61B 5/4094; A61B 5/31; A61B 5/374; A61B 5/6868; A61B 2560/02; A61B 5/28; A61B 5/293; G16H 50/20; G16H 50/30; A61B 5/747

(57) ABSTRACT

Devices, systems, and techniques are described for detecting stroke or seizure with a compact system. For example, a system includes a memory, a plurality of electrodes, and sensing circuitry configured to sense, via at least two electrodes of the plurality of electrodes, electrical signals from a patient, and generate, based on the electrical signals, physiological information. The system may also include processing circuitry configured to receive, from the sensing circuitry, the physiological information, determine, based on the physiological information, a seizure metric indicative of a seizure status of the patient and a stroke metric indicative of a stroke status of the patient, and store the seizure metric and the stroke metric in the memory. A housing may carry the plurality of electrodes and contain both of the sensing circuitry and the processing circuitry.

This application claims the benefit of U.S. Provisional Patent Application Ser. No. 63/071,828, filed Aug. 28, 2020, the entire content of which is incorporated herein by reference.

### TECHNICAL FIELD

This disclosure is directed to medical devices and, more particularly, to systems and methods for detecting seizure and stroke.

### BACKGROUND

Stroke is a serious medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism which may be the result of hemorrhage (e.g., a ruptured blood vessel). During a stroke, the blood supply to an area of a brain may be decreased, which can lead to dysfunction of the brain tissue in that area.

Stroke is the number two cause of death worldwide and the number one cause of disability. Speed to treatment is the critical factor in stroke treatment as 1.9M neurons are lost per minute on average during stroke. Stroke diagnosis and time between event and therapy delivery are the primary barriers to improving therapy effectiveness. Stroke has 3 primary etiologies; i) ischemic stroke (representing approximately 65% of all strokes), ii) hemorrhagic stroke (representing approximately 10% of all strokes), and iii) cryptogenic strokes (includes TIA, representing approximately 25% of all strokes). Strokes can be considered as having neurogenic and/or cardiogenic origins.

A variety of approaches exist for treating patients undergoing a stroke. For example, a clinician may administer anticoagulants, such as warfarin, or may undertake intravascular interventions such as thrombectomy procedures to treat ischemic stroke. As another example, a clinician may administer antihypertensive drugs, such as beta blockers (e.g., Labetalol) and ACE-inhibitors (e.g., Enalapril) or may undertake intravascular interventions such as coil embolization to treat hemorrhagic stroke. Lastly, if stroke symptoms have resolved on their own with negative neurological work-up, a clinician may administer long-term cardiac monitoring (external or implantable) to determine potential cardiac origins of cryptogenic stroke.

Other conditions also affect humans. For example, 65 million people suffer from epilepsy worldwide, with 3.4 million people suffering from epilepsy in the United States. Epilepsy results in approximately 3,400 deaths each year in the United States alone. In some cases, patients may suffer from seizures that are misdiagnosed as epilepsy. For example, 1 out of 4 patients currently diagnosed with epilepsy are ultimately diagnosed as the symptoms originating from some other cause (e.g., seizure of non-brain origin). Epileptic patients may also have other conditions, as approximately one quarter of epileptic patients also suffer from cardiac arrhythmias. Treatments for epilepsy may include lifestyle changes and/or drug therapies.

### SUMMARY

In general, the disclosure is directed to devices, systems, and techniques for detecting seizure (e.g., cardiogenic and/or neurogenic seizures) or stroke via a medical device, e.g., an implantable medical device (IMD) or external medical device, located on the head of a patient. For example, an IMD may include a plurality of electrodes carried by a housing of the device. The IMD may be implanted subcutaneously in a region of the cranium, such as at the back of the head or base of the cranium. From this location, the IMD may be able to record electrical signals from the electrodes carried on the housing. These electrical signals may contain components attributable to brain function and components contributable to cardiac function. The IMD may process the sensed electrical signals to determine stroke metrics indicative of stroke of the patient and seizure metrics indicative of a seizure of the patient. Therefore, the IMD may be able to detect stroke and/or seizure events for the patient from a single device. The IMD may transmit information representative of any detected stroke or seizure to an external device. In some examples, the IMD may transmit information representative of stroke or seizure to another IMD configured to deliver therapy to the patient, such as electrical stimulation therapy and/or drug delivery therapy.

The techniques of this disclosure may provide one or more advantages. For example, it may be beneficial for a single subcutaneously implanted device to be configured to detect stroke and seizure for the patient. In this manner, the IMD may screen the patient for potential strokes or seizures that may inform later treatment. In addition, stroke and seizure may be conditions experienced together for a patient. For example, patients that have epilepsy may be at higher risk of experiencing a stroke. However, these patients may not have other implantable devices capable of identifying stroke or seizure. Therefore, a clinician may be able to implant the IMD that carries the electrodes in a small package to identify any stroke or seizure events. In addition, the IMD may transmit indications of a stroke or seizure to an external device to facilitate treatment for any events that occur. In this manner, the IMD described herein may be used to detect stroke or seizure for a variety of patients, such as patients that have experienced traumatic brain injury, migraines, brain infection, brain tumors, dementia, sleep disorders, or other conditions.

In one example, a system includes a memory; a plurality of electrodes; sensing circuitry configured to: sense, via at least two electrodes of the plurality of electrodes, electrical signals from a patient; and generate, based on the electrical signals, physiological information; processing circuitry configured to: receive, from the sensing circuitry, the physiological information; determine, based on the physiological information, a seizure metric indicative of a seizure status of the patient and a stroke metric indicative of a stroke status of the patient; and store the seizure metric and the stroke metric in the memory; and a housing carrying the plurality of electrodes and containing both of the sensing circuitry and the processing circuitry.

In another example, a method includes sensing, by sensing circuitry and via at least two electrodes of a plurality of electrodes, electrical signals from a patient; generating, by the sensing circuitry and based on the electrical signals, physiological information; receiving, by processing circuitry and from the sensing circuitry, the physiological information; determining, by the processing circuitry and based on the physiological information, a seizure metric indicative of a seizure status of the patient and a stroke metric indicative of a stroke status of the patient; and storing, by the processing circuitry, the seizure metric and the stroke metric in the memory, wherein a housing carries the plurality of electrodes and contains both of the sensing circuitry and the processing circuitry.

In another example, a computer-readable medium comprising instructions that, when executed, cause processing circuitry to control sensing circuitry to sense, via at least two electrodes of a plurality of electrodes, electrical signals from a patient; control the sensing circuitry to generate, based on the electrical signals, physiological information; receive, from the sensing circuitry, the physiological information; determine, based on the physiological information, a seizure metric indicative of a seizure status of the patient and a stroke metric indicative of a stroke status of the patient; and store the seizure metric and the stroke metric in the memory, wherein a housing carries the plurality of electrodes and contains both of the sensing circuitry and the processing circuitry.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A is a conceptual diagram of a system configured to detect stroke or seizure in accordance with examples of the present disclosure.

FIG. 1B is a conceptual diagram of a system configured to detect stroke or seizure in accordance with examples of the present disclosure.

FIG. 1C is a diagram of the 10-20 map for electroencephalography (EEG) sensor measurements.

FIG. 2A depicts a top view of a sensor device in accordance with embodiments of the present technology.

FIG. 2B depicts a side view of the sensor device shown in FIG. 2A in accordance with the present technology.

FIG. 2C depicts a top view of another embodiment of sensor device in accordance with the present technology.

FIG. 2D depicts a side view of another embodiment of a sensor device in accordance with the present technology.

FIG. 2E depicts a side view of another embodiment of a sensor device in accordance with the present technology.

FIG. 2F depicts a side view of another embodiment of a sensor device in accordance with the present technology.

FIG. 2G depicts a top view of another example sensor device in accordance with examples of the present disclosure.

FIG. 2H depicts a top view of another example sensor device in accordance with examples of the present disclosure.

FIG. 2I depicts a top view of another example sensor device that includes electrode extensions in accordance with examples of the present disclosure.

FIGS. 2J, 2K, 2L, 2M, 2N, 2P, and 2Q depict example sensor devices that includes electrode extensions, in accordance with examples of the present disclosure.

FIG. 2Q depicts an example sensor device that includes electrode extensions in conjunction with a patient, in accordance with examples of the present disclosure.

FIGS. 3A, 3B, and 3C depicts another sensor device in accordance with embodiments of the present technology.

FIG. 4 is a block diagram of an example sensor device configured to detect stroke or seizure.

FIG. 5 is a block diagram of an example external device configured to communicate with the sensor device of FIG. 4.

FIG. 6 is a block diagram illustrating an example system that includes an access point, a network, external computing devices, such as a server, and one or more other computing devices, which may be coupled to the IMD, the external device, and the processing circuitry of FIG. 1 via a network, in accordance with one or more techniques described herein.

FIG. 7 is a flow diagram of an example technique for detecting at least one of a stroke or seizure of a patient.

FIG. 8 is a flow diagram of an example technique for timing detection of stroke or seizure based on one or more trigger events.

FIG. 9 is a flow diagram of an example technique for adjusting a seizure detection frequency based on arrhythmia detection.

FIG. 10 is a flow diagram of an example technique for determining a seizure type based on electrocardiogram information.

Many aspects of the present disclosure can be better understood with reference to the following drawings. The components in the drawings are not necessarily to scale. Instead, emphasis is placed on clearly illustrating the principles of the present technology.

### DETAILED DESCRIPTION

This disclosure describes various systems, devices, and techniques for detecting stroke and seizure from a device located on the head of the patient. It can be difficult to determine whether a patient is suffering from a stroke or has suffered from a stroke. Current diagnostic techniques typically involve evaluating a patient for visible symptoms, such as paralysis or numbness of the face, arm, or leg, as well as difficultly walking, speaking, or understanding (e.g., the F.A.S.T visible stroke indication of Face, Arm, Speech, Time to call for emergency help). However, these techniques may result in undiagnosed strokes, particularly more minor strokes that leave patients relatively functional upon cursory evaluation. Even for relatively minor strokes, it is important to treat the patient as soon as possible because treatment outcomes for stroke patients are highly time-dependent. Accordingly, there is a need for improved methods for detecting strokes. However, such treatments may be frequently underutilized and/or relatively ineffective due to the failure to timely identify whether a patient is undergoing or has recently undergone a stroke. This is a particular risk with more minor strokes that leave patients relatively functional upon cursory evaluation.

Similarly, it can be difficult to detect or identify epileptic seizures from other types of seizures. For example, many disorders may manifest themselves as recurrent seizures that are misclassified as epileptic seizures. Syncope is one example condition that may be caused from other physiological issues but is commonly misdiagnosed as epilepsy due to the recurring seizures. Moreover, some patients exhibit physical manifestations of the seizure, such as jerking movements of the arms and legs, other symptoms of a seizure may include temporary confusion, staring, loss of consciousness or awareness, or emotional symptoms such as fear, anxiety, or déjà vu. When patients are experiencing a seizure, the patient may not be able to understand the symptoms or accurately identify what occurred. Moreover, the patient may not be able to obtain or ask for intervention, such as medication. Appropriate diagnosis is thus even more challenging. In some examples, a deep brain stimulation (DBS) device may detect seizure and provide electrical stimulation via electrodes implanted within the brain to prevent or reduce symptoms of seizure. However, such DBS devices require an invasive implantation procedure and may not be appropriate for screening or diagnosis of the patient.

As described herein, a medical device (e.g., an IMD or external medical device wearable by the patient), may be configured to detect stroke and seizure from a location on or near the head of the patient. For example, the IMD may be configured to be implanted subcutaneously without the need for any medical leads. In some examples, instead of leads, the IMD may include a housing that carries multiple electrodes directly on the housing. Using these housing electrodes, the IMD may sense electrical signals from one or more vectors and generate physiological information representative of patient condition. The physiological information may be indicative of brain activity and/or activity of other organs such as the heart. This physiological information may include stroke information and/or seizure information. For example, different sensing circuits may generate the stroke information and the seizure information such that respective filters and amplifiers may extract the relevant components for stroke and seizure detection, respectively. The IMD may then generate, from appropriate physiological information, a stroke metric indicative of whether or not the patient has experienced a stroke and a seizure indicative of whether or not the patient experienced a seizure. For example, the IMD may detect and differentiate different types of seizures such as (1) epileptic seizure (with changes observed on an electroencephalogram (EEG), (2) syncope (a seizure with cardiac origin and no change on EEG), and (3) psychogenic events due to psychological causes with no change on EEG (e.g., also referred to as Psychogenic Non-Epileptic Seizure (PNES), Non-Epileptic Attack Disorder (NEAD) or Psychogenic Pseudo Syncope (PPS). In some examples, the seizure metric may identify which type of seizure has been detected.

The IMD may store the stroke metrics and seizure metrics over time. The IMD may transmit the stroke and seizure metrics to an external device periodically or in response to a trigger event, such as detection of a stroke or seizure being experienced by the patient. In other examples, the IMD may transmit the stroke and/or seizure metric to another IMD or external medical device configured to deliver electrical stimulation therapy and/or drug delivery therapy. In some examples, the IMD may generate the stroke and seizure metrics at different frequencies as needed to provide appropriate monitoring for the patient while conserving power. For example, the IMD may generate seizure metrics at a higher frequency than the stroke metric because a seizure may only last for a few minutes while characteristics of a stroke may last tens of minutes or even hours. In other examples, the IMD may trigger the generation of seizure metrics and/or stroke metrics in response to a trigger event that indicates the risk for seizure or stroke has increased, respectively.

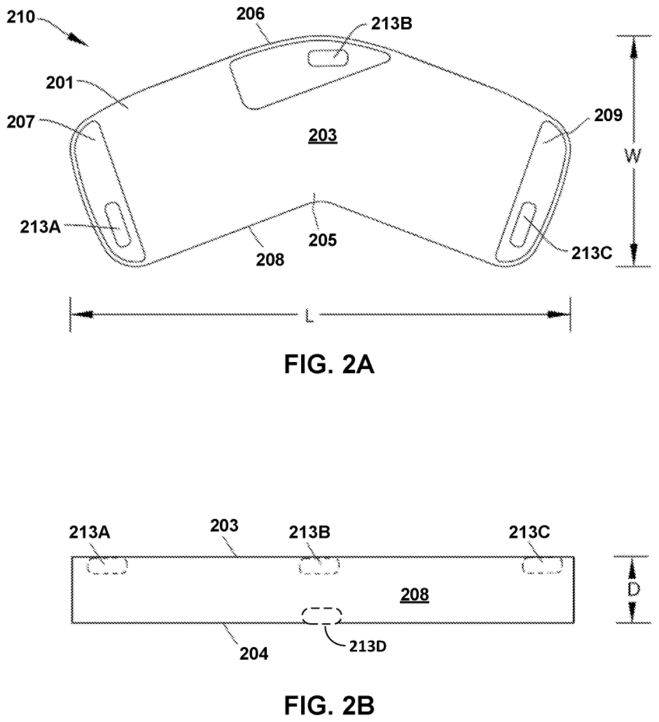

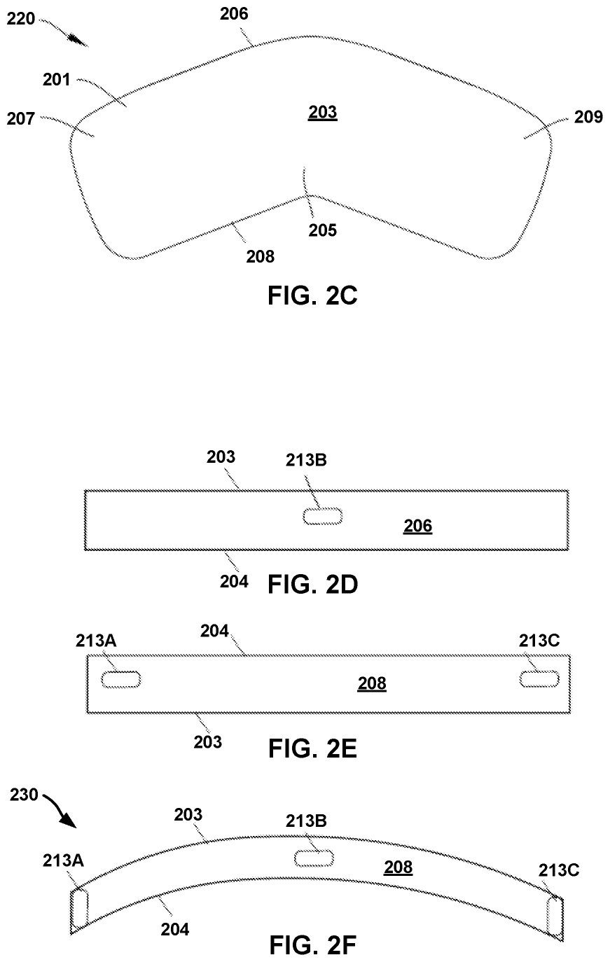

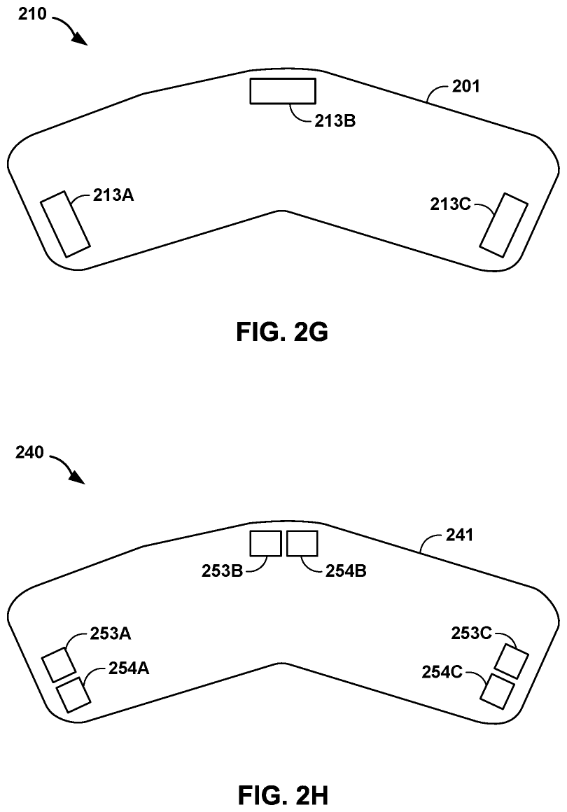

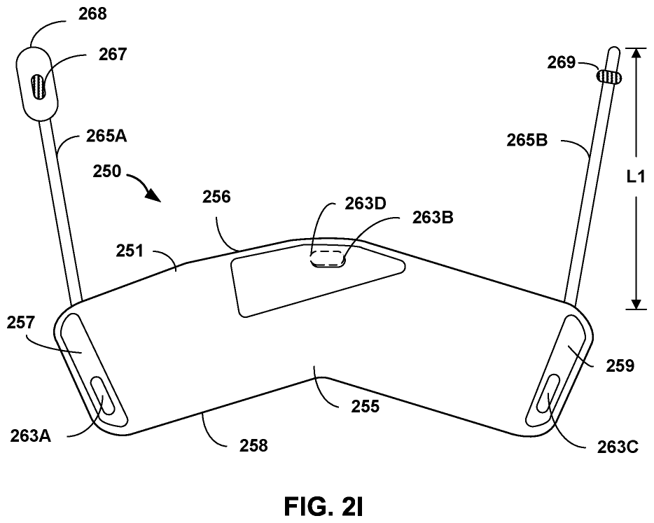

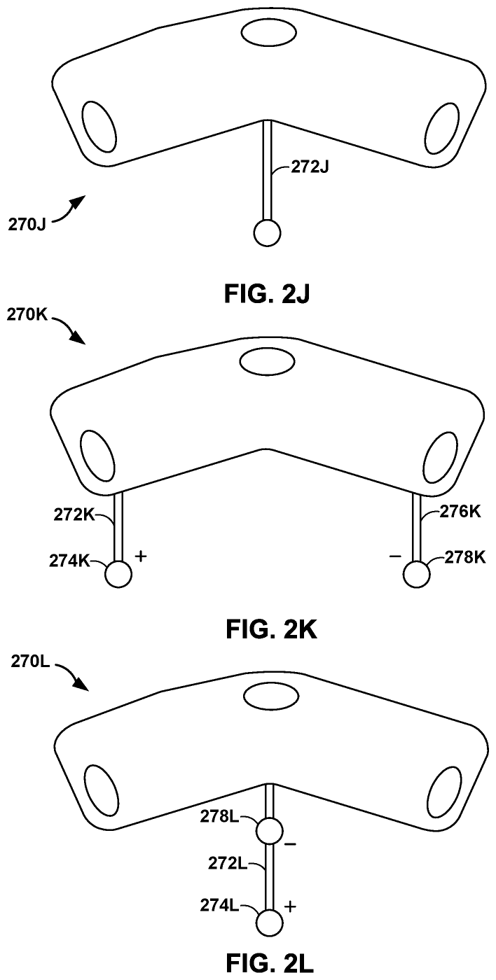

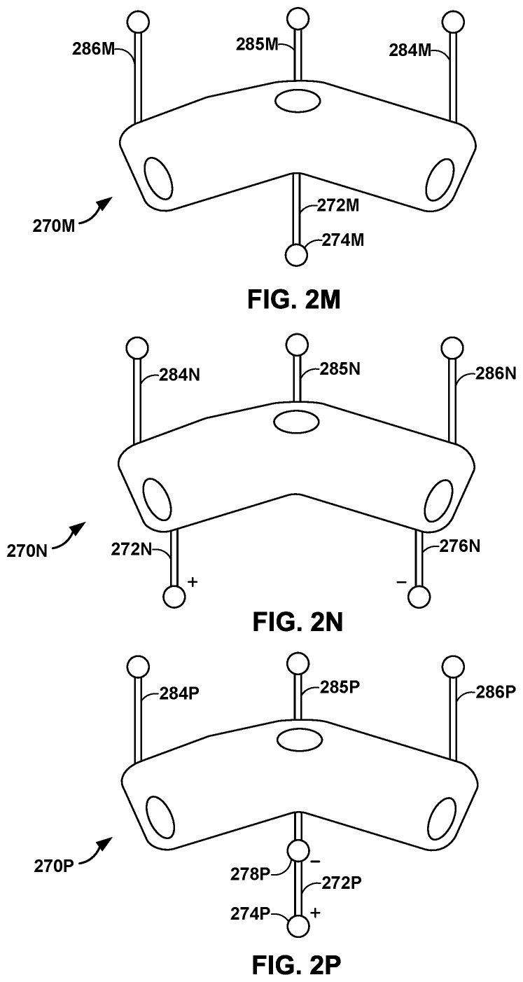

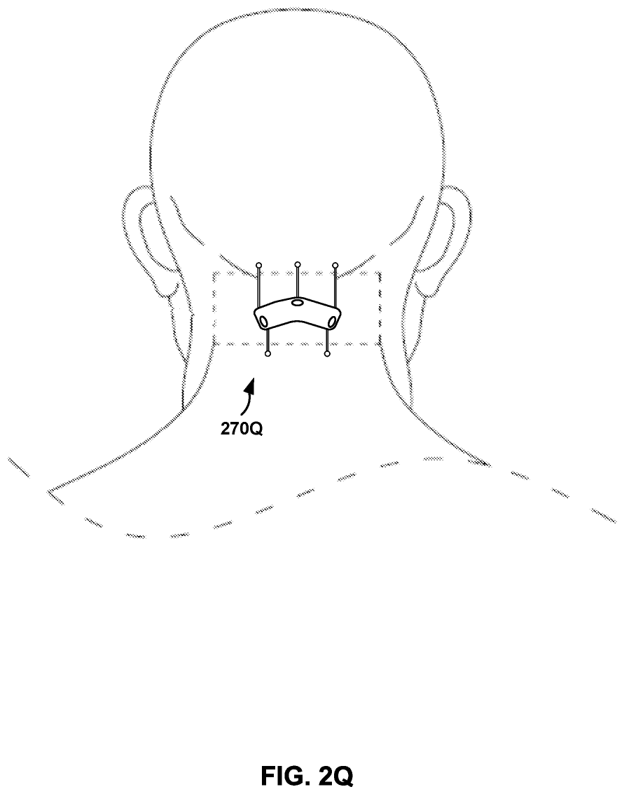

Conventional EEG electrodes are typically positioned over a large portion of a user's scalp. While electrodes in this region are well positioned to detect electrical activity from the patient's brain, there are certain drawbacks. Sensors in this location interfere with patient movement and daily activities, making them impractical for prolonged monitoring. Additionally, implanting traditional electrodes under the patient's scalp is difficult and may lead to significant patient discomfort. To address these and other shortcomings of conventional EEG sensors, embodiments of the present technology include an IMD configured to record electrical signals at a region near the patient's head, such as adjacent a rear portion of the patient's neck or base the patient's skull or near the patient's temple. In these positions, implantation under the patient's skin is relatively simple, and a temporary application of a wearable sensor device (e.g., coupled to a bandage, garment, band, or adhesive member) does not unduly interfere with patient movement and activity. Although primarily described in the context of leadless sensor devices, in some examples, e.g., as described with respect to FIGS. 2I-2Q, a sensor device may include electrode extensions. The electrode extensions may increase a size of a vector for sensing signals via the electrodes, such as brain and cardiac signals, and/or may position electrodes closer to a source of the brain and cardiac signals, which may enhance the sensitivity of algorithms using such signals to detect and/or predict patient conditions.

However, the EEG signals detected via electrodes disposed at or adjacent the back of a patient's neck may include relatively high noise amplitude. For example, the electrical signals associated with brain activity may be intermixed with electrical signals associated with cardiac activity (e.g., ECG signals) or signals including components associated with mechanical activity of the heart and muscle activity (e.g., EMG signals) and artifacts from other electrical sources such as patient movement or external interference. Accordingly, in some embodiments, the sensor data may be filtered or otherwise manipulated to separate the brain activity data (e.g., EEG signals) and ECG signals (or other cardiac signals) from each other and other electrical signals (e.g., EMG signals, etc.).

As described in more detail below, in some embodiments, the physiological information can be analyzed to make a stroke determination or a seizure determination based on one or more thresholds, correlation between signals, or using a classification algorithm, which can itself be derived using machine learning techniques applied to databases of known stroke and/or seizure patient data. The detection algorithm(s) can be passive (involving measurement of a purely resting patient) or active (involving prompting a patient to perform potentially impaired functionality, such as moving particular muscle groups (e.g., raising an arm, moving a finger, moving facial muscles, etc.,) and/or speaking while recording the electrical response), or from an electrical or other stimulus.

Aspects of the technology described herein can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., such as via Bluetooth)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g., a Bluetooth network) on a propagated signal on a propagation medium (e.g., an electromagnetic wave(s), a sound wave) over a period of time, or may be provided on any analog or digital network (packet switched, circuit switched, or other scheme).

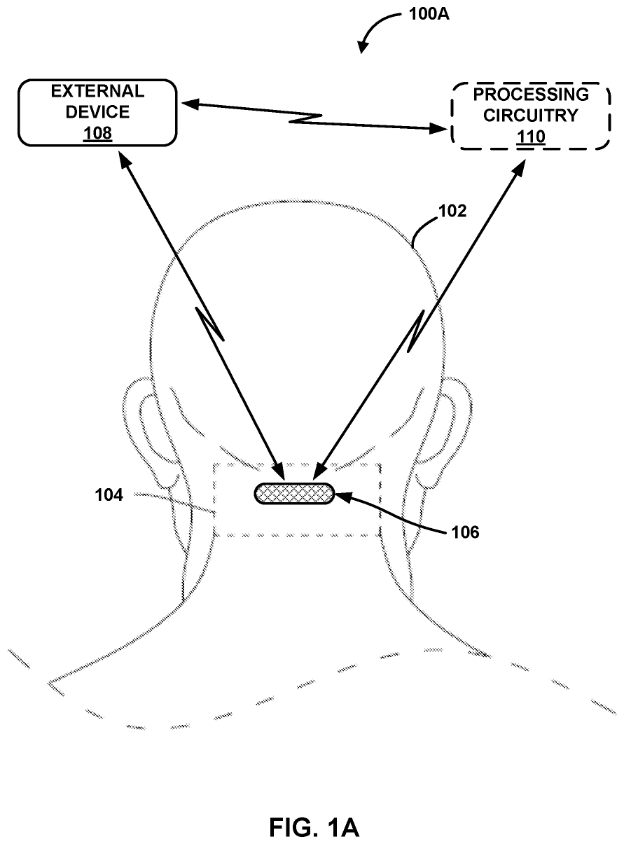

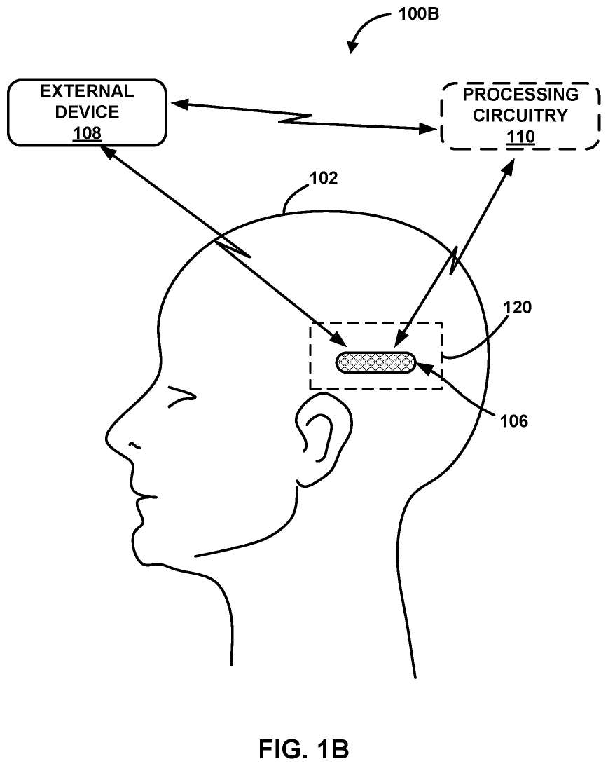

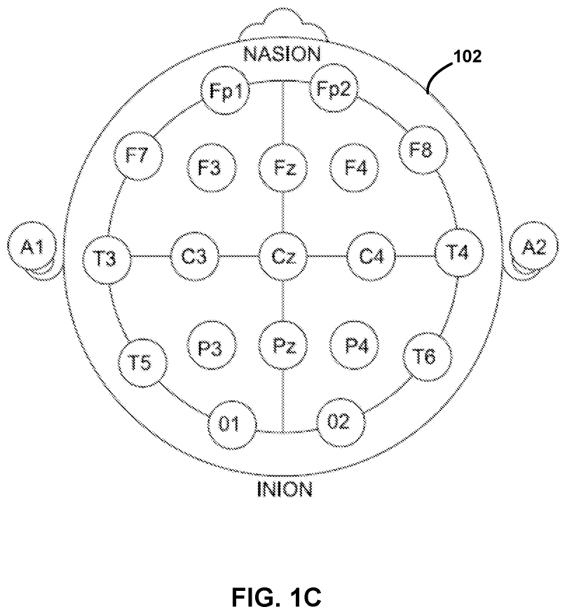

FIG. 1A is a conceptual diagram of a system configured to detect stroke or seizure in accordance with examples of the present disclosure. The example techniques described herein may be used with an implantable medical device (IMD) 106, which may be in wireless communication with at least one of external device 108, processing circuitry 110, and other devices not pictured in FIG. 1A. For example, an external device (not illustrated in FIG. 1A) may include at least a portion of processing circuitry 110, the external device configured for communication with IMD 106, and external device 108. As shown in FIG. 1A, IMD 106 is located in target region 104. Target region 104 can be a rear portion of a user's neck or at the base of the skull. Although IMD 106 may be implanted at a location generally centered with respect to the head, neck, or target region 104, IMD 106 may be implanted in an off-center location in order to obtain desired vectors from the electrodes carried on the housing of IMD 106. In other examples, target region may be located at other positions of patient, such as near the user's temple(s) (e.g., above the ear(s)) and/or over the temporal portion of the skull. IMD 106 can be disposed in target region 104 either via implantation (e.g., subcutaneously) or by being placed over the patient's skin with one or more electrodes of IMD 106 being in direct contact with the patient's skin at or adjacent the target region 104.

While conventional EEG electrodes are placed over the patient's scalp, the present technology advantageously enables recording of clinically useful brain activity data via electrodes positioned at the target region 104 at the rear of the patient's neck or head, or other cranial locations, such as temporal locations, described herein. This anatomical area is well suited to suited both to implantation of IMD 106 and to temporary placement of a sensor device over the patient's skin. In contrast, conventional EEG electrodes positioned over the scalp are cumbersome, and implantation over the patient's skull is challenging and may introduce significant patient discomfort. As noted elsewhere here, conventional EEG electrodes are typically positioned over the scalp to more readily achieve a suitable signal-to-noise ratio for detection of brain activity. However, by using certain digital signal processing, and a special-purpose classifier algorithm, clinically useful brain activity data can be obtained using sensors disposed at the target region 104. Specifically, the electrodes can detect electrical activity that corresponds to brain activity in the P3, Pz, and/or P4 regions (as shown in FIG. 1C).

While conventional approaches to stroke detection utilizing EEG have relied on data from a large number of EEG electrodes, this disclosure describes that clinically useful stroke and seizure determinations can be made utilizing relatively few electrodes, such as via the electrodes carried by IMD 106. For example, IMD 106 may extract features from EEG signals indicative of brain activity or cardiac activity. IMD 106 may then determine whether or not the patient has experienced a stroke or seizure based on these extracted features. In some examples, IMD 106 takes the form of a LINQ™ Insertable Cardiac Monitor (ICM), available from Medtronic plc, of Dublin, Ireland. The example techniques may additionally, or alternatively, be used with a medical device not illustrated in FIG. 1A such as another type of IMD, a patch monitor device, a wearable device (e.g., smart watch), or another type of external medical device.

Clinicians sometimes diagnose a patient (e.g., patient 102) with medical conditions and/or determine whether a condition of patient 102 is improving or worsening based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patient is in a clinic for a medical appointment. However, in some examples, events that may change a condition of a patient, such as administration of a therapy, may occur outside of the clinic. As such, in these examples, a clinician may be unable to observe the physiological markers needed to determine whether an event, such as a seizure or stroke, has changed a medical condition of the patient and/or determine whether a medical condition of the patient is improving or worsening while monitoring one or more physiological signals of the patient during a medical appointment. In the example illustrated in FIG. 1A, IMD 106 is implanted within patient 102 to continuously record one or more physiological signals of patient 102 over an extended period of time.

In some examples, IMD 106 includes a plurality of electrodes. The plurality of electrodes is configured to detect signals that enable processing circuitry of IMD 106 to determine current values of stroke metrics and seizure metrics associated with the brain and/or cardiovascular functions of patient 102. In some examples, the plurality of electrodes of IMD 106 are configured to detect a signal indicative of an electric potential of the tissue surrounding the IMD 106. Moreover, IMD 106 may additionally or alternatively include one or more optical sensors, accelerometers, impedance sensors, respiration sensors, temperature sensors, chemical sensors, light sensors, pressure sensors, and acoustic sensors, in some examples. Such sensors may detect one or more physiological parameters indicative of a patient condition. In some examples, IMD 106 may be implanted such that a sensor, such as an impedance sensor, detects characteristic of a skin such as an impedance sensor configured to detect changes in the skin that may correlate with temperature changes or perspiration that can occur as a result of one or more types of seizures.

External device 108 may be a hand-held computing device with a display viewable by the user and an interface for providing input to external device 108 (e.g., a user input mechanism). For example, external device 108 may include a small display screen (e.g., a liquid crystal display (LCD) or a light emitting diode (LED) display) that presents information to the user. In addition, external device 108 may include a touch screen display, keypad, buttons, a peripheral pointing device, voice activation, or another input mechanism that allows the user to navigate through the user interface of external device 108 and provide input. If external device 108 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 108 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device.

When external device 108 is configured for use by the clinician, external device 108 may be used to transmit instructions to IMD 106. Example instructions may include requests to set electrode combinations for sensing and any other information that may be useful for programming into IMD 106. The clinician may also configure and store operational parameters for IMD 106 within IMD 106 with the aid of external device 108. In some examples, external device 108 assists the clinician in the configuration of IMD 106 by providing a system for identifying potentially beneficial operational parameter values.

Whether external device 108 is configured for clinician or patient use, external device 108 is configured to communicate with IMD 106 and, optionally, another computing device (not illustrated by FIG. 1A), via wireless communication. External device 108, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies). In some examples, external device 108 is a smartphone of patient 102 and/or a watch or other wearable computing device, which may communicate with sensor device 106, e.g., via Bluetooth™. In some examples, external device 108 is configured to communicate with a computer network, such as the Medtronic CareLink® Network developed by Medtronic, plc, of Dublin, Ireland. For example, external device 108 may send data, such as data received from IMD 106, to another external device such as a smartphone, a tablet, or a desktop computer, and the other external device may in turn send the data to the computer network. In other examples, external device 108 may directly communicate with the computer network without an intermediary device.

Processing circuitry 110, in some examples, may include one or more processors that are configured to implement functionality and/or process instructions for execution within IMD 106. For example, processing circuitry 110 may be capable of processing instructions stored in a storage device. Processing circuitry 110 may include, for example, microprocessors, graphical processing units (GPUs), tensor processing units (TPUs), digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 110 may include any suitable structure, whether in hardware, software, firmware, or any combination thereof, to perform the functions ascribed herein to processing circuitry 110.

Processing circuitry 110 may represent processing circuitry located within any one or both of IMD 106 and external device 108. In some examples, processing circuitry 110 may be entirely located within a housing of IMD 106. In other examples, processing circuitry 110 may be entirely located within a housing of external device 108. In other examples, processing circuitry 110 may be located within any one or combination of IMD 106, external device 108, and another device or group of devices that are not illustrated in FIG. 1A. As such, techniques and capabilities attributed herein to processing circuitry 110 may be attributed to any combination of IMD 106, external device 108, and other devices that are not illustrated in FIG. 1A.

Medical device system 100A of FIG. 1A is an example of a system configured to collect electrical signals and generate stroke metrics and seizure metrics according to one or more techniques of this disclosure. In some examples, processing circuitry 110 includes sensing circuitry configured to generate physiological information from the sensed electrical signal of patient 102. In one example, an electrical signal is sensed via one or more electrode combinations of IMD 106. An electrical signal is representative of electrical activity of the brain, heart, or other physiological functions as measured by electrodes implanted within the body. For example, the sensed electrical signals may include features representative of heart function such as P-waves (depolarization of the atria), R-waves (depolarization of the ventricles), and T-waves (repolarization of the ventricles), among other events. Information relating to the aforementioned events, such as time separating one or more of the events, may be applied for a number of purposes, such as to determine whether an arrhythmia is occurring and/or predict whether an arrhythmia is likely to occur. Cardiac signal analysis circuitry, which may be implemented as part of processing circuitry 110, may perform signal processing techniques to extract information indicating the one or more parameters of the cardiac signal. In other examples, the sensed electrical signals may include features representative of brain function, such as amplitudes of frequencies in one or more frequency bands, such as alpha bands, beta bands, or gamma bands. Brain signal analysis circuitry, which may be implemented as part of processing circuitry 110 may perform various processing circuitry to extract these brain features from the sensed electrical signals. In some examples, the sensed signals may be surrogates for brain and cardiac electrical signals (e.g., EEG or ECG signals). Pulsatile signals sensed from the scalp vasculature correspond to ventricular contractions and ECG R-waves, albeit with a slight timing delay.

In some examples, IMD 106 includes one or more accelerometers. An accelerometer of IMD 106 may collect an accelerometer signal which reflects a measurement of any one or more of a motion of patient 102, a posture of patient 102 and a body angle of patient 102. In some cases, the accelerometer may collect a three-axis accelerometer signal indicative of patient 102's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 102 along a vertical axis, the lateral axis accelerometer signal vector may represent an acceleration of patient 102 along a lateral axis, and the frontal axis accelerometer signal vector may represent an acceleration of patient 102 along a frontal axis. In some cases, the vertical axis substantially extends along a torso of patient 102 when patient 102 from a neck of patient 102 to a waist of patient 102, the lateral axis extends across a chest of patient 102 perpendicular to the vertical axis, and the frontal axis extends outward from and through the chest of patient 102, the frontal axis being perpendicular to the vertical axis and the lateral axis.

IMD 106 may measure a set of parameters including an impedance (e.g., subcutaneous impedance measured via electrodes depicted in FIGS. 2A-2Q, an intrathoracic impedance or an intracardiac impedance) of patient 102, a respiratory rate of patient 102 during night hours, a respiratory rate of patient 102 during day hours, a heart rate of patient 102 during night hours, a heart rate of patient 102 during day hours, an atrial fibrillation (AF) burden of patient 102, a ventricular rate of patient 102 while patient 102 is experiencing AF, or any combination thereof. Processing circuitry 110 may analyze any one or more of the set of parameters in order to determine whether or not the patient is experiencing stroke or seizure, and may indicate an efficacy of a treatment program administered to patient 102. In some examples, pulsatile signals sensed optically or mechanically, e.g., via the electrodes, an optical sensor, accelerometer, pressure sensor, impedance sensor, or heart sound sensor, from the scalp vasculature may provide a surrogate for an ECG or other cardiac electrical activity signal. In some examples, the treatment program may include treatment delivered by one or more medical devices such as ICDs with intravascular or extravascular leads, pacemakers, CRT-Ds, neuromodulation devices, LVADs, implantable sensors, orthopedic devices, or drug pumps. Additionally, or alternatively, the treatment program may include in-clinic treatments administered by medical professionals, prescribed pharmaceutical regimens, treatments administered by one or more external medical devices, or any combination thereof. In any case, processing circuitry 110 may determine the efficacy of the treatment program by determining a time in which the treatment program is administered (e.g., including a time in which the treatment program begins and/or a time in which the treatment program ends) and analyzing values of any one or combination of the set of parameters relative to the time in which the treatment program is administered. Alternatively, in some examples, processing circuitry 110 may determine the efficacy of a treatment program by evaluating one or more parameters on a rolling basis in order to determine whether the one or more parameters have changed over a period of time.

In some examples, one or more sensors (e.g., electrodes, motion sensors, optical sensors, temperature sensors, or any combination thereof) of IMD 106 may generate a signal that indicates a parameter of a patient. In some examples, the signal that indicates the parameter includes a plurality of parameter values, where each parameter value of the plurality of parameter values represents a measurement of the parameter at a respective interval of time. The plurality of parameter values may represent a sequence of parameter values, where each parameter value of the sequence of parameter values are collected by IMD 106 at a start of each time interval of a sequence of time intervals. For example, IMD 106 may perform a parameter measurement in order to determine a parameter value of the sequence of parameter values according to a recurring time interval (e.g., every day, every night, every other day, every twelve hours, every hour, or any other recurring time interval). In this way, IMD 106 may be configured to track a respective patient parameter more effectively as compared with a technique in which a patient parameter is tracked during patient visits to a clinic, since IMD 106 is implanted within patient 102 and is configured to perform parameter measurements according to recurring time intervals without missing a time interval or performing a parameter measurement off schedule. Processing circuitry 110 may determine these different parameters separately from the seizure or stroke metrics or determine the seizure or stroke metrics based at least partially on one or more other parameter measurements.

IMD 106 may be referred to as a system or device. In one example, IMD 106 may include a memory, a plurality of electrodes carried by the housing of IMD 106, sensing circuitry configured to sense, via at least two electrodes of the plurality of electrodes, electrical signals from patient 10 and generate, based on the electrical signals, physiological information. IMD 106 may also include processing circuitry configured to receive, from the sensing circuitry, the physiological information and determine, based on the physiological information, a seizure metric indicative of a seizure status of the patient and a stroke metric indicative of a stroke status of the patient. The processing circuitry may be configured to then store the seizure metric and the stroke metric in the memory. The housing of IMD 106 carries the plurality of electrodes and contains, or houses, both of the sensing circuitry and the processing circuitry. In this manner, IMD 106 may be referred to as a leadless sensing device because the electrodes are carried directly by the housing instead of by any leads that extend from the housing. In some examples, however, sensor device 106 may include one or more sensing leads extending therefrom and into the tissue of the patient; such lead(s) may be employed instead of or in addition to the electrodes of sensor device 106 (e.g., such as electrode extensions depicted in FIG. 2I-2Q), and may perform any of the functions attributed herein to the electrodes.

The physiological data can include electrical brain activity data and/or electrical heart activity data. In some examples, the plurality of electrodes are configured to detect brain activity data corresponding to activity in at least one of a P3, Pz, or P4 brain region, which is at the back of the head or upper neck region as shown in FIG. 1C. In this manner, the housing of IMD 106 may be configured to be disposed at or adjacent to a rear portion of a neck or skull of patient 102. The housing of IMD 106 may be configured to be implanted within patient 104, such as implanted subcutaneously. In other examples, the housing of IMD 106 may be configured to be disposed on an external surface of skin of patient 102.

In some examples, IMD 106 may include a single sensing circuitry configured to generate, from the sensed electrical signals, information that includes both the electrical brain activity data (e.g., electroencephalogram (EEG) data) and the electrical heart activity data (e.g., electrocardiogram (ECG) data or cardiac contraction). In other examples, the processing circuitry of IMD 106 may include separate hardware that generates different information from the sensed electrical signals. For example, IMD 106 may include first circuitry configured to generate the electrical brain activity from the electrical signals and second circuitry different from the first circuitry and configured to generate the electrical heart activity data from the electrical signals. Even with the first and second circuitry configured to generate different information, or data, in some examples, sensed electrical signals may be conditioned or processed by one or more electrical components (e.g., filters or amplifiers) prior to being processed by the first and second circuitry. In some examples, electrical brain activity data may include features, such as spectral features, indicative of the strength of signals in various frequency bands or at various frequencies. In this manner, IMD 106 may generate a seizure metric based on this electrical brain activity data. In some examples, electrical heart activity data may include features such as the timing and/or amplitude of P-waves, R-waves, or any other features representative of heart function.

Each of the stroke metrics and the seizure metrics may be indicative of the likelihood (or risk) that patient 102 has experienced, or is experiencing, a stroke or a seizure, respectively. For example, each stroke metric and seizure metric may include a numerical value representative of the probability that patient 102 has experienced a stroke or a seizure. IMD 106 may then compare the metric to a respective threshold or monitor a relative change in the metric value over time to determine whether or not a stroke or seizure has occurred. In other examples, the stroke and/or seizure metric may be a binary value that indicates no event occurred or that an event did occur. In some examples, IMD 106 may generate each stroke metric and/or seizure metric based on sensed data other than the sensed electrical signals from the carried electrodes on the housing of IMD 106.

In one example, IMD 106 may include one or more accelerometers within the housing. The accelerometer may be configured to generate motion data representative of motion of patient 102. IMD 106 may then be configured to determine, based on the physiological data that includes the motion data, the seizure metric and the stroke metric. For example, body motion, or lack thereof, may be indicative of a type of seizure experienced by patient 102. In one example, IMD 106 may utilize the signal generated by the accelerometer, at least in part, classify a seizure as a motor onset or non-motor onset seizure. For motor onset seizures, IMD 106 may further utilize the accelerometer signal to classify a motor onset seizure as a tonic-clonic seizure or some other motor seizure type. As another example, certain body motions or behaviors (e.g., patterns of motion) may be indicative of stroke. In one example, the processing circuitry of IMD 106 may be configured to determine, based on the motion data, that patient 102 has fallen. In response to determining that patient 102 has fallen, the processing circuitry may be configured to determine, or inform, the stroke metric based on the determination that the patient has fallen. IMD 106 may correlate the motion data for the fall detection with the stroke metric based on time. For example, IMD 106 may consider the time window from the detection of the fall to other factors associated with the stroke metric. In this manner, if the fall detection occurs within a predetermined or calculated time window from one or more other factors associated with the stroke metric, the fall detection may be included or weighted more because it may be predictive of stroke. The fall detection may be a factor in the stroke metric because a stroke may cause a patient to fall. Therefore, in combination with other features extracted from sensed electrical signals, IMD 106 may determine from the fall indication that the stroke metric indicates detection of a stroke. In other examples, IMD 106 may determine that a characteristic of the motion data exceeds a threshold. The threshold may be an acceleration value indicative of a fall, for example. Responsive to determining that the characteristic of the motion data exceeds the threshold, the processing circuitry of IMD 106 may determine at least one of the seizure metric or the stroke metric. For seizure, for example, a frequency of the motion data exceeding a frequency threshold may be indicative of body movement from a seizure.

In some examples, the physiological information generated from the sensed electrical signals may include ECG information. IMD 106 may extract various features from the ECG information, such as heart rate, heart rate variability, etc. IMD 106 may determine, based on the seizure metric, that patient 102 has experienced a seizure and select, based on the ECG information and from a plurality of seizure types, one seizure type representative of the seizure experienced by the patient. For example, seizure types may include single seizure, stroke induced seizure, epileptic seizure, absence seizures, tonic-clonic or convulsive seizures, atonic seizures, clonic seizures, tonic seizures, and myoclonic seizures. In some examples, IMD 106 may also determine the seizure type based on accelerometer data, temperature data, or any other parameter extracted from one or more sensors.

IMD 106 may generate the seizure metrics and the stroke metrics at the same or different frequencies. In some examples, these frequencies may refer to the frequency at which the sensing circuitry generates appropriate information from which the stroke or seizure metric is determined. In other examples, IMD 106 may continually generate physiological information from which both stroke and seizure metrics can be determined. However, the frequency may refer to how often the processing circuitry generates the stroke or seizure metric from the physiological information. The seizure detection frequency may be different than the stroke detection frequency due to the duration and/or effects from a seizure or stroke. For example, a seizure may only last for a few minutes, but stroke may last for hours. Therefore, the seizure detection frequency is greater than the stroke detection frequency in some examples. In this manner, timers may be used to trigger the detection of stroke and seizure. Such variation in detection frequency may enable IMD 106 to conserve power and only monitor for stroke or seizure when appropriate.

In other examples, trigger events for seizure or stroke detection may be identified from various sensed data. For example, the processing circuitry of IMD 106 may be configured to determine, based on an ECG signal of the physiological information, an arrhythmia of a heart of the patient 102. For some patients, arrhythmias may cause, or be caused by, seizures. Responsive to determining the arrhythmia, IMD 106 may thus increase a seizure detection frequency that controls determination of the seizure metric and determine the seizure metric according to the seizure detection frequency. In this manner, IMD 106 may vary the monitoring frequency for seizure based on the presence of any arrythmias in the heart.

FIG. 1B is a conceptual diagram of a system 100B configured to detect stroke or seizure in accordance with examples of the present disclosure. System 100B may be substantially similar to system 100A of FIG. 1A. However, system 100B may be configured to be implanted in target region 120 which is located on the side of the head posterior of the temple of patient 102, e.g., above the ear and/or over the temporal portion of the cranium. IMD 106 implanted at target region 120 may be configured to generate stroke metrics and seizure metrics based on electrical signals sensed in this area. In such examples, the electrodes of sensor device 106 may detect electrical activity that corresponds to brain activity in the T3 region (as shown in FIG. 1C), or T4 region if implanted on the other side of the patient's head, or both of two or more sensor devices are implanted bilaterally at temporal regions. In some examples, IMD 106 may need to employ different filters or other processing or signal conditioning techniques than those at target region 104 due to different types of noise at target region 120, such as muscle activity due to mandible movement or other types of electrical activity. In other examples, IMD 106 may be configured to determine seizure metrics and stroke metrics from other areas of the head of patient 102 that may be outside of target regions 104 and 120.

FIG. 1C is a diagram of the 10-20 map for electroencephalography (EEG) sensor measurements. As shown in FIG. 1C, various locations on the head of patient 12 may be targeted using the electrodes carried by IMD 106. At the back of the head, such as in target region 104 of FIG. 1A, IMD 106 may sense electrical signals at least one of P3, Pz or P4 At the side of the head, such as in target region 120 of FIG. 1B, IMD 106 may sense electrical signals at at least one of F7, T3, or T5 adjacent the left hemisphere of the brain. In addition or alternatively, IMD 106 may sense electrical signals at at least one of F8, T4, or T6 adjacent the right hemisphere of the brain.

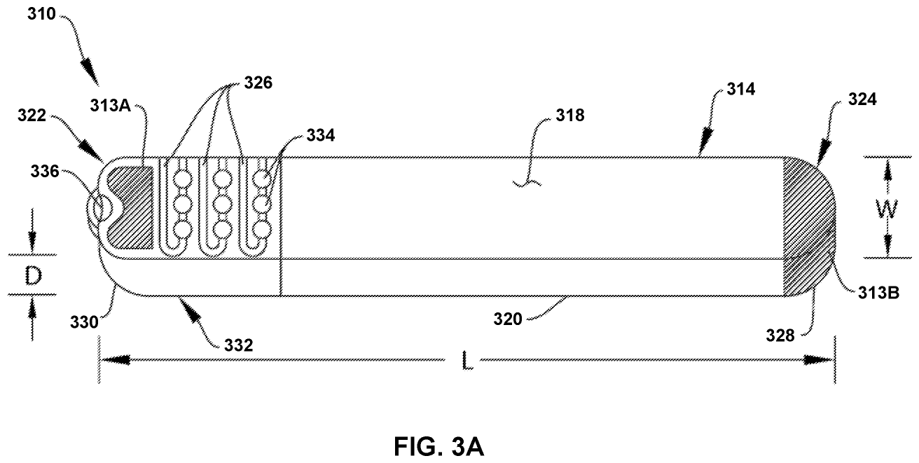

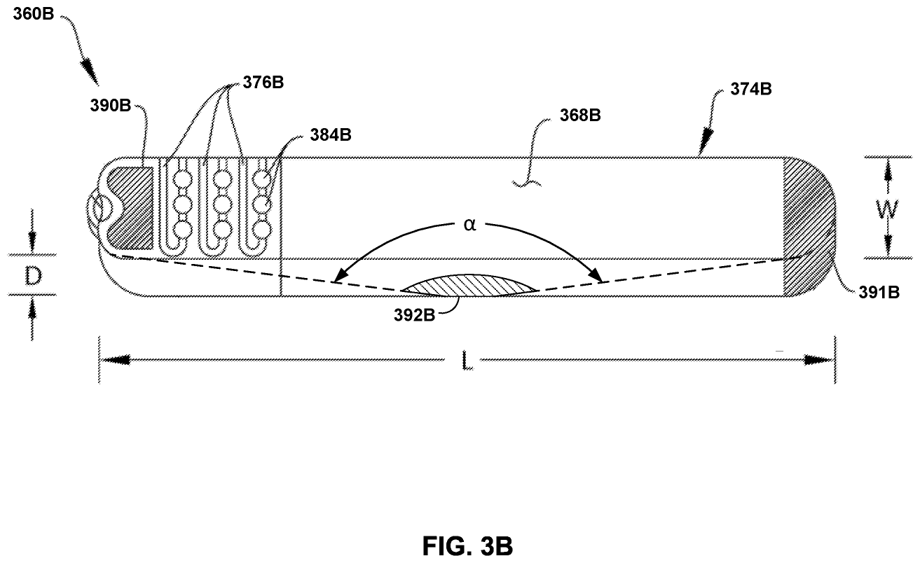

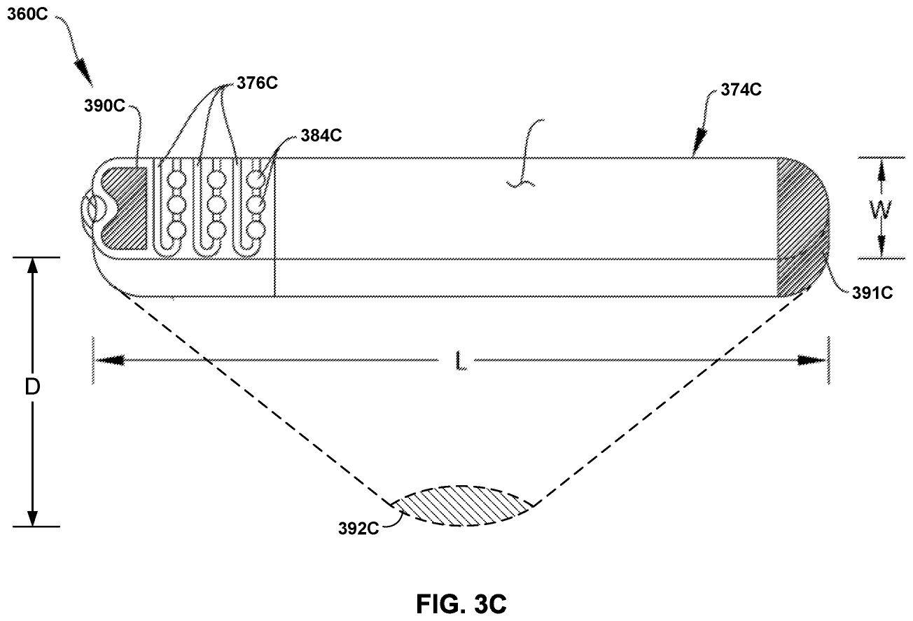

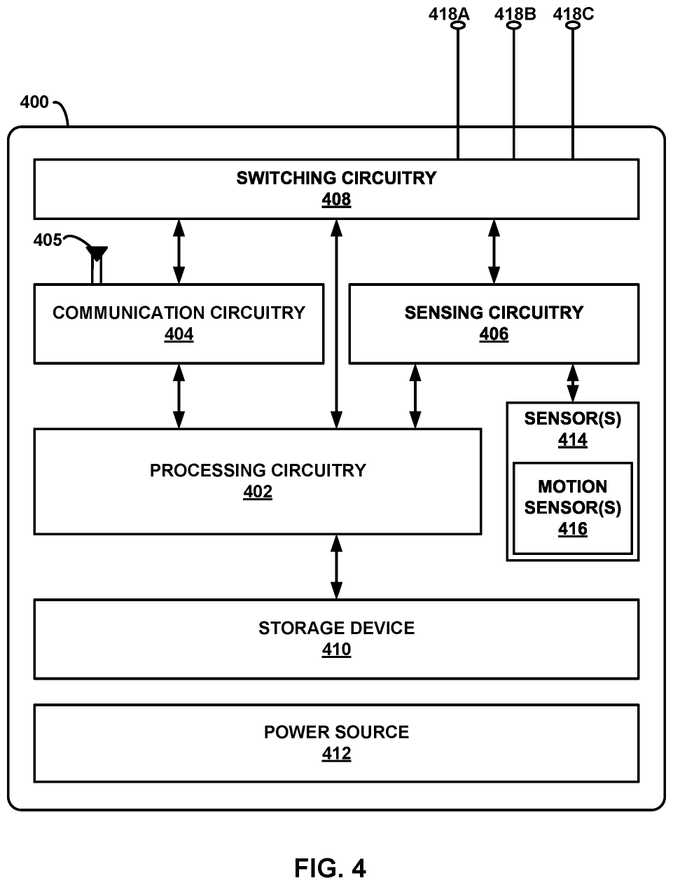

FIG. 2A depicts a top view of a sensor device (e.g., an implantable medical device) in accordance with embodiments of the present technology. FIG. 2B depicts a side view of sensor device 210 shown in FIG. 2A in accordance with the present technology. FIG. 2A illustrates a plan view of an example sensor device 210. In some embodiments, the sensor device 210 can include some or all of the features of, and be similar to, IMD 106 described above with respect to FIGS. 1A and 1B and/or the IMDs 310, 360B, 360C, or 400 described below with respect to FIGS. 3A-3C and 4, and can include additional features as described in connection with FIG. 2A. In the illustrated example, the sensor device 210 includes a housing 201 that carries a plurality of electrodes 213A, 213B, 213C, 213D (collectively “electrodes 213”) therein. Although four electrodes are shown for sensor device 210, in other examples, only two or three electrodes may be carried or more than four electrodes may be carried by housing 201. In some examples, each electrode may be configured to sense both ECG or other cardiac signals and EEG signals. In other examples, each electrode may be wired to only detect a single type of signal, such as ECG or EEG. In this manner, sensing circuitry may utilize distinct electrodes for sensing EEG and ECG. In such a configuration, two different and independently wired electrodes may be located at the location of each of electrodes 213 shown in FIG. 2A. In operation, electrodes 213 can be placed in direct contact with tissue at the target site (e.g., with the user's skin if placed over the user's skin, or with subcutaneous tissue if the sensor device 210 is implanted). Housing 201 additionally encloses electronic circuitry located inside the sensor device 210 and protects the circuitry (e.g., processing circuitry, sensing circuitry, communication circuitry, sensors, and a power source) contained therein from body fluids. In various embodiments, electrodes 213 can be disposed along any surface of the sensor device 210 (e.g., anterior surface, posterior surface, left lateral surface, right lateral surface, superior side surface, inferior side surface, or otherwise), and the surface in turn may take any suitable form.

In the example of FIGS. 2A and 2B, housing 201 can be a biocompatible material having a relatively planar shape including a first major surface 203 configured to face towards the tissue of interest (e.g., to face anteriorly when positioned at the back of the patient's neck) a second major surface 204 opposite the first, and a depth D or thickness of housing 201 extending between the first and second major surfaces. Housing 201 can define a superior side surface 206 (e.g., configured to face superiorly when sensing device 210 is implanted in or at the patient's head or neck) and an opposing inferior side surface 208. Housing 201 can further include a central portion 205, a first lateral portion (or left portion) 207, and a second lateral portion (or right portion) 209. Electrodes 213 are distributed about housing 201 such that a central electrode 213B is disposed within the central portion 205 (e.g., substantially centrally along a horizontal axis of the device), a back electrode 213D is disposed on inferior side surface, a left electrode 213A electrode is disposed within the left portion 207, and a right electrode 213C is disposed within the right portion 209. As illustrated, housing 201 can define a boomerang or chevron-like shape in which the central portion 205 includes a vertex, with the first and second lateral portions 207 and 209 extending both laterally outward and from the central portion 205 and also at a downward angle with respect to a horizontal axis of the device. In other examples, housing 201 may be formed in other shapes which may be determined by desired distances or angles between different electrodes 213 carried by housing 201.

The configuration of housing 201 can facilitate placement either over the user's skin in a bandage-like form or for subcutaneous implantation. As such, a relatively thin housing 201 can be advantageous. Additionally, housing 201 can be flexible in some embodiments, so that housing 201 can at least partially bend to correspond to the anatomy of the patient's neck (e.g., with left and right lateral portions 207 and 209 of housing 201 bending anteriorly relative to the central portion 205 of housing 201).

In some examples, housing 201 can have a length L of from about 15 to about 50 mm, from about 20 to about 30 mm, or about 25 mm. Housing 201 can have a width W from about 2.5 to about 15 mm, from about 5 to about 10 mm, or about 7.5 mm. In some embodiments, housing 201 can have a thickness of the thickness is less than about 10 mm, about 9 mm, about 8 mm, about 7 mm, about 6 mm, about 5 mm, about 4 mm, or about 3 mm. In some embodiments, the thickness of housing 201 can be from about 2 to about 8 mm, from about 3 to about 5 mm, or about 4 mm. Housing 201 can have a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc. In some embodiments, housing 201 can have dimensions suitable for implantation through a trocar introducer or any other suitable implantation technique.

As illustrated, electrodes 213 carried by housing 201 are arranged so that all three electrodes 213 do not lie on a common axis. In such a configuration, electrodes 213 can achieve a variety of signal vectors, which may provide one or more improved signals, as compared to electrodes that are all aligned along a single axis. This can be particularly useful in a sensor device 210 configured to be implanted at the neck or head while detecting electrical activity in the brain. In some embodiments, this electrode configuration also provides for improved cardiac ECG sensitivity by integrating 3 potential signal vectors. In some examples, processing circuitry may create virtual signal vectors through a weighted sum or two or more physical signal vectors, such as the physical signal vectors available from electrodes 213 of sensor device 210 or the electrodes of any other sensor device described herein.

In the example shown in FIG. 2B, all electrodes 213 are located on the first major surface 203 and are substantially flat and outwardly facing. However, in other examples one or more electrodes 213 may utilize a three-dimensional configuration (e.g., curved around an edge of the device 210). Similarly, in other examples, such as that illustrated in FIG. 2B, one or more electrodes 213 may be disposed on the second major surface opposite the first. The various electrode configurations allow for configurations in which electrodes 213 are located on both the first major surface and the second major surface. In other configurations, such as that shown in FIG. 2B, electrodes 213 are only disposed on one of the major surfaces of housing 201. Electrodes 213 may be formed of a plurality of different types of biocompatible conductive material (e.g., stainless steel, titanium nitride, platinum iridium, iridium, or alloys thereof), and may utilize one or more coatings such as titanium nitride or fractal titanium nitride. In some embodiments, the material choice for electrodes can also include materials having a high surface area (e.g., to provide better electrode capacitance for better sensitivity) and roughness (e.g., to aid implant stability). Although the example shown in FIGS. 2A and 2B includes four electrodes 213, in some embodiments the sensor device 210 can include 1, 2, 3, 4, 5, 6, or more electrodes carried by housing 201.

FIG. 2C depicts a top view of another example of sensor device 220 in accordance with the present technology. FIG. 2C illustrates sensor device 220 which is substantially similar to sensor device 210, but sensor device 220 includes electrodes 213 which are not exposed along the first major surface 203 of housing 201. Instead, electrodes 213 can be exposed along superior and inferior side surfaces (e.g., facing superiorly and inferiorly when implanted at or on a patient's neck), as shown in FIGS. 2D and 2E. FIG. 2F illustrates sensor device 230 which is substantially similar to sensor devices 210 and 220, but housing 201 is constructed to have a curved configuration, and in which the electrodes can be place along the superior and/or inferior side surfaces of housing 201. In some embodiments, a curved configuration can improve patient comfort and more readily conform to the anatomy of the patient's neck region. In some examples, any of sensor devices 210, 220, or 230 may be flexible in order to conform to the anatomy of the patient at the desired implant or external surface location. Additionally, examples that include electrode extensions, e.g., as depicted in FIGS. 2I-2Q, are inherently flexible, allowing conformance to neck and/or cranial anatomy. In some examples, sensor device 220 and/or sensor device 230 may be implanted at a location generally centered with respect to the thorax, the head, e.g., back or temporal regions, neck, or another target region. In some examples, sensor device 220 and/or sensor device 230 may be placed on an external surface of skin of a patient.

In operation, electrodes 213 are used to sense electrical signals (e.g., EEG signals and/or ECG signals) which may be submuscular or subcutaneous. The sensed electrical signals may be stored in a memory of the sensor device 210, and signal data may be transmitted via a communications link to another device (e.g., external device 108 of FIG. 1A). The sensed electrical signals may be time-coded or otherwise correlated with time data, and stored in this form, so that the recency, frequency, time of day, time span, or date(s) of a particular signal data point or data series (or computed measures or statistics based thereon) may be determined and/or reported. In some examples, electrodes 213 may additionally or alternatively be used for sensing any bio-potential signal of interest, such as an electrocardiogram (ECG), intracardiac electrogram (EGM), electromyogram (EMG), or a nerve signal, from any implanted location. These data may be time-coded or time-correlated, and stored in that form, in the manner described above with respect to EEG signal data.

FIGS. 2G and 2H depict top views of devices in accordance with examples of the present disclosure. FIG. 2G depicts housing 201 of sensor device 210, which includes electrodes 213A-213C arranged at the perimeter of housing 201. Each of electrodes 213A-213C may be configured to receive raw signals including cardiac and brain components. Sensor device 210 may include circuitry configured to filter the raw signals received by electrodes 213A-213C to generate cardiac signals and brain signals, e.g., ECG and EEG signals. In some examples, this circuitry may be located outside of sensor device 210.

FIG. 2H depicts housing 241 of sensor device 240, which includes electrodes 253A-253C and 254A-254C. Electrodes 253A and 254A together may be referred to as a segmented electrode. Similarly, electrodes 253B and 254B may be referred to as a segmented electrode, and electrodes 253C and 254C may be referred to as a segmented electrode. Insulative material may separate the conductive portions (e.g., electrodes 253A and 254A) of a segmented electrode.

Circuitry may be configured to generate a first cardiac, e.g., ECG, signal based on a differential signal received at electrodes 253A and 253B, generate a second cardiac signal based on a differential signal received at electrodes 253B and 253C, and/or generate a third cardiac signal based on a differential signal received at electrodes 253C and 253A. Likewise, the circuitry may be configured to generate a first brain, e.g., EEG, signal based on a differential signal received at electrodes 254A and 254B, generate a second brain signal based on a differential signal received at electrodes 254B and 254C, and/or generate a third brain signal based on a differential signal received at electrodes 254C and 254A.

FIG. 2I depicts a top view of another example sensor device 250, which includes electrodes 263A-236D, 267, and 269. Each of electrodes 263A-236D, 267, and 269 may be configured to receive raw signals including cardiac and brain, e.g., ECG and EEG, components. Sensor device 250 may include circuitry configured to filter the raw signals received by electrodes 263A-236D, 267, and 269 to generate cardiac signals and brain signals, e.g., ECG and EEG signals. Sensor device 250 may also include circuitry configured to measure impedance of tissue via electrodes 263A-236D, 267, and 269.

In the example of FIG. 2I, sensor device 250 include a housing 251, which includes a superior side surface 256, an opposing inferior side surface 258, a central portion 255, a first lateral portion (or left portion) 257, and a second lateral portion (or right portion) 259. Electrodes 263 are distributed about housing 251 such that a central electrode 263B is disposed within the central portion 255 (e.g., substantially centrally along a horizontal axis of the device), a left electrode 263A is disposed within the left portion 257, and a right electrode 263C is disposed within the right portion 259.

Sensor device 250 further includes electrode extensions 265A and 265B (collectively “electrode extensions 265”). As illustrated in FIG. 2I, electrode extension 265A includes a paddle 268 such that one or more electrodes 267 are distributed on paddle 268. Electrode extension 265B includes one or more ring electrodes 269. In some examples, electrode extensions 265 may be connected to a housing 256 of sensor device 250 via header pins. In some examples, electrode extensions 265 may be permanently attached to housing 256 of sensor device 250. The number and types of electrode extensions 265, electrodes on such extensions, and electrodes on housing 251 may differ from that illustrated by FIG. 2I in other examples.

In some examples, electrode extensions 265 can have a length L1 of from about 15 to about 50 mm, from about 20 to about 30 mm, or about 25 mm. One or more electrode extensions 265 may provide sensor device 250 larger sensing vectors for sensing signals via electrodes. The larger (longer) sensing vectors that include one or more electrodes on one or more extensions may facilitate improved signal quality relative to smaller (shorter) sensing vectors.

Electrode extensions 265 may be inherently flexible, allowing conformance to neck and/or cranial anatomy. Additionally, the length and flexibility of one or more electrode extensions 265 may allow electrodes on the extension to advantageously be positioned proximate to certain brain structures or locations, vascular structures, or other anatomical structures or locations, which may also facilitate improved signal quality, e.g., when the signal originates from or is affected by the structure. For example, electrode extensions 265A and 265B can extend superiorly from sensor device 250 for enhanced brain signal sensing and detection. Improved signal quality may result in improved performance of algorithms for predicting or detecting patient conditions using such signals. In examples in which one or more electrode extensions 265 are implanted, the extension may be tunneled under the scalp to a position one or more electrodes on the extension at a desired location of the cranium.

FIGS. 2J-2P depict example sensor devices 270J, 270K, 270L, 270M, 270N, and 270P (collectively “sensor devices 270”) that include electrode extensions 272J-272P, 276K, 276N, 284M-284P, 285M-285P, and 286M-286P, in accordance with examples of the present disclosure. Sensor devices 270 may be similar in other aspects to the sensor devices illustrated and described with respect to FIGS. 1A-2I, e.g., may include electronics within a housing, and electrodes on the housing. The electrode extensions shown in FIGS. 2J-2P may also be referred to as leads. The electrodes on electrode extensions 272J-272P, 276K, 276N, 284M-284P, 285M-285P, and 286M-286P may be ring electrodes or paddle electrodes, as examples.

FIGS. 2J-2L show examples of electrode extensions 272J-272L and 276K that can be positioned to extend from sensor devices 270J-270L in a first direction, and FIGS. 2M-2P show examples of electrode extensions 272M-272P and 276N that can be positioned to extend from sensor devices 270M-270P in the first direction and electrode extensions 284M-284P, 285M-285P, and 286M-286P that can be positioned to extend from sensor devices 270M-270P in a second direction that is opposite from the first direction. In some examples, the first direction may be an inferior direction, e.g., towards the neck and shoulders of the patient, and the second direction may be a superior direction, e.g., towards the upper cranium and scalp of the patient. For example, a first electrode extension may be positioned to extend towards a first temporal region, and a second electrode extension may be positioned to extend towards a second temporal region. Electrode extensions 272J-272P and 276K shown in FIGS. 2J-2P can extend towards the neck and shoulders for enhanced cardiac signal sensing and detection. Electrode extensions 284M-284P, 285M-285P, and 286M-286P shown in FIGS. 2M-2P can be positioned to extend superiorly toward the upper cranium and scalp for enhanced brain signal sensing.

FIG. 2J shows an example of single electrode extension 272J that extends from the center of sensor device 270J. FIG. 2K shows an example of two electrode extensions 272K and 276K that extend from opposing ends of sensor device 270K. Electrode 274K on electrode extension 272K may have a positive polarity, and electrode 278K on electrode extension 276K may have a negative polarity, or the polarities of the electrodes may be reversed, such that the electrodes have opposite polarities. FIG. 2L shows an example of single electrode extension 272L that extends from the center of sensor device 270L. There are two electrodes 274L and 278L with opposing polarities on single electrode extension 272L. Thus, sensor devices 270K and 270L shown in FIGS. 2K and 2L may be configured to receive a differential signal via electrodes 274K, 278K, 274L, and 278L on one or more electrode extensions 272K, 276K, and 272L. In the example of FIG. 2J, a generally vertical or non-horizontal sensing vector may be formed between the electrode on extension 272J and an electrode on a housing of sensor device 270J, and in the example of FIG. 2L, a generally vertical sensing vector may be formed between electrodes 274L and 278L on extension 272L. In the example of FIG. 2K, a generally horizontal sensing vector may be formed between electrode 274K on extension 272K and electrode 278K on extension 276K.

Each of sensor devices 270M-270P shown in FIGS. 2M-2P includes one or more electrode extensions 272M-272P and 276N that extend in a first direction and three electrode extensions 284M-284P, 285M-285P, and 286M-286P that extend in a second direction that is opposite from the first direction. FIG. 2M shows single electrode extension 272M that extends in the first direction from the center of sensor device 270M. FIG. 2N shows two electrode extensions 272N and 276N that extend in the first direction from opposing ends of sensor device 270N. FIG. 2P shows single electrode extension 272P that extends from the center of sensor device 270P, where single electrode extension 272P includes two electrodes 274P and 278P of opposing polarities.

FIG. 2Q shows sensor device 270Q on the back of a patient's neck. In the illustrated example, sensor device 270Q is configured similarly to sensor device 270N of FIG. 2N. However, any of the sensor devices including extensions described with respect to FIGS. 2I-2P may be positioned in the manner illustrated by sensor device 270Q in FIG. 2Q. Additionally, any of the sensor devices including extensions described with respect to FIGS. 2I-2P may be positioned at other locations described herein, such as temporally as illustrated with respect to FIG. 1B.

Such sensor devices may include one or more extensions extending in a first, inferior direction, toward the neck or shoulders of the patient. Extensions extending in this first direction may position electrodes to facilitate cardiac signal, e.g., ECG, sensing. Such sensor devices may include one or more extensions extending in a second, superior direction, opposite the first direction, toward the upper cranium and scalp of the patient. Extensions extending in this second direction may facilitate brain signal, e.g., EEG, sensing. Each extension may include one or more electrodes to provide one or more sensing vectors of one or more orientations with another electrode on the same extension, a different extension, or a housing of the sensor device.

FIGS. 3A-3C depicts an example sensor devices 310, 360B, and 360C in accordance with embodiments of the present technology. In some examples, sensor device 310 can include some or all of the features of IMDs 106 or 400, sensor devices 210, 220, and 230, described herein in accordance with embodiments of the present technology, and can include additional features as described in connection with FIG. 3A. In the example shown in FIG. 3A, sensor device 310 may be embodied as a monitoring device having housing 314, proximal electrode 313A and distal electrode 313B (individually or collectively “electrode 313” or “electrodes 313”). Housing 314 may further comprise first major surface 318, second major surface 320, proximal end 322, and distal end 324. Housing 314 encloses electronic circuitry located inside sensor device 310 and protects the circuitry contained therein from body fluids. Electrical feedthroughs provide electrical connection of electrodes 313. In an example, sensor device 310 may be embodied as an external monitor, such as patch that may be positioned on an external surface of the patient, or another type of medical device (e.g., instead of as an ICM), such as described further herein.

In the example shown in FIG. 3A, sensor device 310 is defined by a length “L,” a width “W,” and thickness or depth “D.” sensor device 310 may be in the form of an elongated rectangular prism wherein the length L is significantly larger than the width W, which in turn is larger than the depth D. In one example, the geometry of sensor device 310—in particular, a width W being greater than the depth D—is selected to allow sensor device 310 to be inserted under the skin of the patient using a minimally invasive procedure and to remain in the desired orientation during insertion. For example, the device shown in FIG. 3A includes radial asymmetries (notably, the rectangular shape) along the longitudinal axis that maintains the device in the proper orientation following insertion. For example, in one example the spacing between proximal electrode 313a and distal electrode 313B may range from 30 millimeters (mm) to 55 mm, 35 mm to 55 mm, and from 40 mm to 55 mm and may be any range or individual spacing from 25 mm to 60 mm. In-some examples, the length L may be from 30 mm to about 70 mm. In other examples, the length L may range from 40 mm to 60 mm, 45 mm to 60 mm and may be any length or range of lengths between about 30 mm and about 70 mm. In addition, the width W of first major surface 18 may range from 3 mm to 10 mm and may be any single or range of widths between 3 mm and 10 mm. The thickness of depth D of sensor device 310 may range from 2 mm to 9 mm. In other examples, the depth D of sensor device 310 may range from 2 mm to 5 mm and may be any single or range of depths from 2 mm to 9 mm. In addition, sensor device 310 according to an example of the present disclosure is has a geometry and size designed for ease of implant and patient comfort. Examples of sensor device 310 described in this disclosure may have a volume of 3 cc or less, 2 cc or less, 1 cc or less, 0.9 cc or less, 0.8 cc or less, 0.7 cc or less, 0.6 cc or less, 0.5 cc or less, or 0.4 cc or less, any volume between 3 and 0.4 cc, or any volume less than 0.4 cc. In addition, in the example shown in FIG. 3A, proximal end 322 and distal end 324 are rounded to reduce discomfort and irritation to surrounding tissue once inserted under the skin of the patient.

In the example shown in FIG. 3A, once inserted within the patient, the first major surface 318 faces outward, toward the skin of the patient while the second major surface 320 is located opposite the first major surface 318. Consequently, the first and second major surfaces may face in directions along a sagittal axis of patient, and this orientation may be consistently achieved upon implantation due to the dimensions of sensor device 310. Additionally, an accelerometer, or axis of an accelerometer, may be oriented along the sagittal axis.

Proximal electrode 313A and distal electrode 313B are used to sense electrical signals (e.g., EEG signals, ECG signals, other brain and/or cardiac signals, or impedance) which may be submuscular or subcutaneous. Electrical signals may be stored in a memory of sensor device 310, and signal data may be transmitted via integrated antenna 326 to another medical device, which may be another implantable device or an external device, such as external device 108 (FIG. 1A). In some examples, electrodes 313A and 313B may additionally or alternatively be used for sensing any bio-potential signal of interest, such as an electrocardiogram (ECG), intracardiac electrogram (EGM), electromyogram (EMG), or a nerve signal, from any implanted location.

In the example shown in FIG. 3A, proximal electrode 313A is in close proximity to the proximal end 322, and distal electrode 313B is in close proximity to distal end 324. In this example, distal electrode 313B is not limited to a flattened, outward facing surface, but may extend from first major surface 318 around rounded edges 328 or end surface 330 and onto the second major surface 320 so that the electrode 313B has a three-dimensional curved configuration. In the example shown in FIG. 3A, proximal electrode 313A is located on first major surface 318 and is substantially flat, outward facing. However, in other examples proximal electrode 313A may utilize the three-dimensional curved configuration of distal electrode 313B, providing a three-dimensional proximal electrode (not shown in this example). Similarly, in other examples distal electrode 313B may utilize a substantially flat, outward facing electrode located on first major surface 318 similar to that shown with respect to proximal electrode 313A. The various electrode configurations allow for configurations in which proximal electrode 313A and distal electrode 313B are located on both first major surface 318 and second major surface 320. In other configurations, such as that shown in FIG. 3A, only one of proximal electrode 313A and distal electrode 313B is located on both major surfaces 318 and 320, and in still other configurations both proximal electrode 313A and distal electrode 313B are located on one of the first major surface 318 or the second major surface 320 (e.g., proximal electrode 313A located on first major surface 318 while distal electrode 313B is located on second major surface 320). In another example, sensor device 310 may include electrodes 313 on both first major surface 318 and second major surface 320 at or near the proximal and distal ends of the device, such that a total of four electrodes 313 are included on sensor device 310. Electrodes 313 may be formed of a plurality of different types of biocompatible conductive material (e.g., stainless steel, titanium nitride, platinum, iridium, or alloys thereof), and may utilize one or more coatings such as titanium nitride or fractal titanium nitride. Although the example shown in FIG. 3A includes two electrodes 313, in some embodiments sensor device 310 can include 3, 4, 5, or more electrodes carried by the housing 314.

In the example shown in FIG. 3A, proximal end 322 includes a header assembly 332 that includes one or more of proximal electrode 313A, integrated antenna 326, anti-migration projections 334, or suture hole 336. Integrated antenna 326 is located on the same major surface (i.e., first major surface 318) as proximal electrode 313a and is also included as part of header assembly 332. Integrated antenna 326 allows sensor device 310 to transmit or receive data. In other examples, integrated antenna 326 may be formed on the opposite major surface as proximal electrode 313A, or may be incorporated within the housing 314 of sensor device 310. In the example shown in FIG. 3A, anti-migration projections 334 are located adjacent to integrated antenna 326 and protrude away from first major surface 318 to prevent longitudinal movement of the device. In the example shown in FIG. 3A anti-migration projections 334 includes a plurality (e.g., six or nine) small bumps or protrusions extending away from first major surface 318. As discussed above, in other examples anti-migration projections 334 may be located on the opposite major surface as proximal electrode 313A or integrated antenna 326. In addition, in the example shown in FIG. 3A header assembly 332 includes suture hole 336, which provides another means of securing sensor device 310 to the patient to prevent movement following insert. In the example shown, suture hole 336 is located adjacent to proximal electrode 313A. In one example, header assembly 332 is a molded header assembly made from a polymeric or plastic material, which may be integrated or separable from the main portion of sensor device 310.

FIG. 3B shows a third electrode 392B at a midpoint between electrodes 390B and 391B. The dimension D of housing 374B of sensor device 360B can be increased to adjust the angle α to obtain a more orthogonal orientation for the triangular configuration of electrodes 390B-392B. In some examples, sensor device 360B may have the same shape and dimensions as sensor device 310, except that electrode 392B is added to the side surface or back surface of housing 374B to create a triangle-shaped electrode configuration. In addition, FIG. 3C shows sensor device 360C with an extended third dimension D. Third electrode 392C is positioned at a corner to create a triangular-shaped electrode configuration with electrodes 390C and 391C. Dimension D can be designed to achieve specific angles for the triangular configuration of electrodes 390C-392C.

FIG. 4 is a block diagram of an example IMD 400 configured to detect stroke or seizure. IMD 400 may be an example of any of IMD 106 or sensor devices 210, 220, 230, 240, 250, 270, 310, 360B, or 360C. In the illustrated example, 1 MB 400 includes electrodes 418A-418C (collectively, “electrodes 418”), antenna 405, processing circuitry 402, sensing circuitry 406, communication circuitry 404, storage device 410, switching circuitry 408, sensors 414 including motion sensor(s) 416, and power source 412. Although not illustrated in FIGS. 3A-3C, sensors 414 may include one or more light detectors.

Processing circuitry 402 may include fixed function circuitry and/or programmable processing circuitry. Processing circuitry 402 may include any one or more of a microprocessor, a GPU, a TPU, a controller, a DSP, an ASIC, an FPGA, or equivalent discrete or analog logic circuitry. In some examples, processing circuitry 402 may include multiple components, such as any combination of one or more microprocessors, one or more GPUs, one or more TPUs, one or more controllers, one or more DSPs, one or more ASICs, or one or more FPGAs, as well as other discrete or integrated logic circuitry. The functions attributed to processing circuitry 402 herein may be embodied as software, firmware, hardware or any combination thereof. Processing circuitry 402 may be an example of or component of processing circuitry 110 (FIGS. 1A and 1B), and may be processing circuitry of any of sensor devices 106, 210, 220, 230, 240, 250, 270, 310, 360A, and 360B.

Sensing circuitry 406 and communication circuitry 404 may be selectively coupled to electrodes 418A-418C via switching circuitry 408, as controlled by processing circuitry 402. Sensing circuitry 406 may monitor signals from electrodes 418A-418C in order to monitor electrical activity of the brain (e.g., to produce an EEG) and/or hearth (e.g., to product an ECG) from which processing circuitry 402 may generate stroke metrics and seizure metrics. Sensing circuitry 406 may also sense physiological characteristics such as subcutaneous tissue impedance, the impedance being indicative of at least some aspects of patient 102's respiratory patterns and the EMG or ECG being indicative of at least some aspects of patient 102's cardiac patterns. Sensing circuitry 406 also may monitor signals from sensors 414, which may include motion sensor(s) 416, and any additional sensors, such as light detectors or optical sensors, pressure sensors, or acoustic sensors, that may be positioned on or in sensor device 400.

In some examples, a subcutaneous impedance signal collected by IMD 400 may indicate a respiratory rate and/or a respiratory intensity of patient 102 and an EMG collected by IMD 400 may indicate a heart rate of patient 102 and an atrial fibrillation (AF) burden of patient 102 or other arrhythmia. In some examples, a respiration component may additionally (using a blended sensor technique) or alternatively be sensed in other signals, such as a motion sensor signal, optical signal, or as a component (e.g., baseline shift) of the cardiac signal sensed via electrodes 418. Sensing circuitry 406 also may monitor signals from sensors 414, which may include motion sensor(s) 416, and any additional sensors, such as light detectors or pressure sensors, that may be positioned on IMD 400. Sensors 414 may also or alternatively detect heart sounds, respiration (e.g., rate or timing), impedance, or blood pressure. Therefore, sensors 414 may also or alternatively include sensors such as one or more microphones, pressure sensors, electrodes, etc. IMD 400 may utilize any of these sensors 414 to determine one or more physiological signals of the patient that may be employed to detect a certain type of seizure or a stroke. In some examples, sensing circuitry 406 may include one or more filters and amplifiers for filtering and amplifying signals received from one or more of electrodes 418A-418C and/or motion sensor(s) 42. In some examples, sensing circuitry 406 may include separate hardware (e.g., separate circuits) configured to condition and process sensed electrical signals from which seizure metrics and stroke metrics are generated. In this manner each separate circuit may perform one or more filters and amplifiers configured to extract relevant features or signal components from the sensed electrical signals. Moreover, processing circuitry 402 may selective control each separate circuit depending on whether a seizure or stroke metric should be generated.

Communication circuitry 404 may include any suitable hardware, firmware, software or any combination thereof for communicating with another device, such as external device 108 or another IMD or sensor, such as a pressure sensing device. Under the control of processing circuitry 402, communication circuitry 404 may receive downlink telemetry from, as well as send uplink telemetry to, external device 108 or another device with the aid of an internal or external antenna, e.g., antenna 405. In some examples, communication circuitry 404 may receive downlink telemetry from, as well as send uplink telemetry to, external device 108 or another device via tissue conductance communication (TCC) using two or more of electrodes 418, e.g., as selected by processing circuitry 402 via switching circuitry 408. In addition, processing circuitry 402 may communicate with a networked computing device via an external device (e.g., external device 108) and a computer network, such as the Medtronic CareLink® Network developed by Medtronic, plc, of Dublin, Ireland. In some examples, communication circuitry 404 may be configured to leverage tissue conductance communication (TCC) for communicating within IMD 400 to between other devices.

A clinician or other user may retrieve data from IMD 400 using external device 108, or by using another local or networked computing device configured to communicate with processing circuitry 402 via communication circuitry 404. The clinician may also program parameters of IMD 400 using external device 108 or another local or networked computing device.

In some examples, storage device 410 may be referred to as a memory and include computer-readable instructions that, when executed by processing circuitry 402, cause IMD 400 and processing circuitry 402 to perform various functions attributed to IMD 400 and processing circuitry 402 herein. Storage device 410 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 410 may also store data generated by sensing circuitry 406, such as physiological information, or data generated by processing circuitry 402, such as stroke metrics and seizure metrics.

Power source 412 is configured to deliver operating power to the components of IMD 400. Power source 412 may include a battery and a power generation circuit to produce the operating power. In some examples, the battery is rechargeable to allow extended operation. In some examples, recharging is accomplished through proximal inductive interaction between an external charger and an inductive charging coil within external device 108. Power source 412 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

As described herein, IMD 400 may be configured to sense electrical signals and generate stroke metrics indicative of whether or not patient 102 has experienced a stroke. In some examples, the processing circuitry 402 is configured to analyze data from one or more electrode combinations using electrodes 418 to extract brain activity data and to discard or reduce any contribution from heart or muscle activity. In some examples, the electrodes 418 are configured to be disposed over the patient's skin. In such embodiments, the electrodes 418 can include protrusions (e.g., microneedles or other suitable structures) configured to at least partially penetrate the patient's skin so as to improve detection of subcutaneous electrical activity.

In some examples, sensing circuitry 406 senses a brain signal via electrodes 418. The brain signal may represent the electrical activity of the brain, and may be an EEG. Processing circuitry 402 may determine parameter values from the brain signal, such values determined based on magnitudes of the signal in one or more frequency bands. Sensing circuitry 406 may include filters and other circuitry to isolate the brain signal of interest.

In some examples, sensing circuitry 406 senses a cardiac signal, and processing circuitry 402 may determine parameter values from the cardiac signal. Example parameter values as described herein, such as heart rate or heart rate variability, may be determined based on detection of occurrence of cardiac beats in the cardiac signal. Sensing circuitry 406 may be configured to sense a variety of different signals within which cardiac beats may be identified and values of cardiac parameters may be determined.

For example, sensing circuitry 406 may be configured to sense a cardiac signal representing the electrical activity (e.g., depolarizations and repolarizations) of the heart, such as a subcutaneous ECG signal, via electrodes 418. As another example, sensing circuitry 406 may be configured to sense a cardiac signal representing mechanical activity of the heart via electrodes 418. A component of a signal sensed via electrodes 418, e.g., on or under the scalp of the patient, may vary based on vibration, blood flow, or impedance changes associated with cardiac contractions. Filtering to isolate this component may include 0.5 to 3 Hz bandpass filtering, although other filtering types, ranges, and cutoffs are possible. In some examples, sensing circuitry 406 may be configured to sense a cardiac signal representing mechanical activity of the heart via other sensors 414, such as optical sensors, pressure sensors, or motion sensors 416.

For example, sensing circuitry 406 and/or processing circuitry 402 may detect cardiac pulses via an optical sensor. Processing circuitry 402 may determine heart rate or heart rate variability based on the detection of cardiac pulses via the optical sensor, e.g., in combination with an ECG signal or in the absence of an ECG signal, such as if ECG signal quality is poor. An optical sensor signal may additionally or alternatively be used for other purposes, such as to determine blood oxygenation, local tissue perfusion, or blood pressure, any of which may be useful for detection or prediction of stroke and/or discrimination of ischemic and hemorrhagic stroke.

One or more electrodes 418 may be positioned, e.g., during implantation of sensor device 400, to facilitate sensing of a cardiac signal via the electrodes. In some examples, sensor device 400 may include one or more electrode extensions 265, 272, 276, 284, 285, 286 to facilitate positioning of one or more electrodes 418, e.g., via tunneling under the scalp, at desired locations for sensing the brain and/or cardiac signals. Desired locations for sensing brain and cardiac signals using electrodes 418 may be determined prior to implantation of sensor device 406 for a particular patient using external sensing equipment, such as standard multi-electrode ECG and EEG equipment, either on the particular patient, or experimentally on a number of subjects. In some examples, the one or more housing-based electrodes 418 of sensor device 400 are positioned at a desired location for sensing a brain signal and the one or more extension-based electrodes 418 are positioned at a desired location for sensing a cardiac signal, or vis-a-versa. With reference to FIG. 1C, example locations for positioning an electrode for sensing cardiac signals include P3, PQ3, PQ7, F3, F2, AF3, or C2.

In some examples, processing circuitry 402 may utilize both electrical, e.g., ECG, and pulsatile cardiac signals in an integrated fashion for the detection, prediction, and/or classification of conditions. In some examples, such integration may result in an “enhanced” ECG signal. For example, processing circuitry 402 may identify features within an ECG signal based on the timing of pulses in a pulsatile signal. In some examples, processing circuitry 402 may account for a delay in pulsatile timing relative to the ECG in such integration.

For example, an optical sensor signal (e.g., a photoplethysmographic signal) can be used as a timing base for ensemble averaging or other means to improve the signal-to-noise ratio for a cardiac signal. The optical sensor signal can therefore be considered a surrogate cardiac signal and/or be used to derive an enhanced cardiac signal, which may be particularly useful when the ECG has poor quality. A first or second derivative of an optical sensor signal can be used as a trigger for ensemble averaging, e.g., the ECG signal, by, for example, determining the time associated with a maximum/minimum value of the first or second derivative and/or a zero-crossing of the first or second derivative. Sharp, high-frequency points can be used as trigger points to increase the resolution of the ensemble signal, whereas lower-frequency trigger points may smear or distort the ensemble average. The cardiac waveforms that are aligned with the trigger points can be stored and averaged to generate the ensemble signal.

Processing circuitry 402 may be configured to calculate physiological characteristics relating to one or more electrical signals received from the electrodes 418, such as stroke metrics. For example, processing circuitry 402 may be configured to algorithmically determine the presence or absence of a stroke (via generation of a stroke metric) or other neurological condition from the electrical signal. In certain examples, processing circuitry 402 may make a stroke determination for each electrode 418 (e.g., channel) or may make a stroke determination using electrical signals acquired from two or more selected electrodes 418.

IMD 400 may also be configured to sense electrical signals and generate seizure metrics indicative of whether or not patient 102 has experienced a seizure. For example, processing circuitry 402 may be configured to analyze physiological information received from sensing circuitry 406 (e.g., EEG information). Processing circuitry 402 may search for one or more features in the physiological information that are indicative of one or more types of seizures. For example, processing circuitry 402 may identify frequency bands that include oscillations or amplitudes that exceed respective thresholds. In other examples, processing circuitry 402 may apply one or more machine learning algorithms or other algorithms to the physiological information to identify when the patient's physiological information is indicative of a seizure. In some examples, processing circuitry 402 may normalize patient data to baseline characteristics. For example, since each patient is different, the sensed data from a patient may be normalized to baseline measurements so that the machine learning algorithms can interpret data from different patients similarly and identify a specific metric, or risk score, for each patient.

In some examples, processing circuitry 402 may employ patient movement information as a part of seizure detection and stroke detection. For example, motion sensor 416 may include one or more accelerometers configured to detect patient movement. Processing circuitry 402 or sensing circuitry 406 may determine whether or not a patient has fallen based on the patient movement data collected via the accelerometer. Fall detection can be particularly valuable when assessing potential stroke patients, as a large percentage of patients admitted for ischemic or hemorrhagic stroke have been found to have had a significant fall within 15 days of the stroke event. Accordingly, in some embodiments, the processing circuitry 402 can be configured to initiate monitoring of brain activity via the electrodes 418 upon fall detection using the accelerometer. In some examples, the sensing performed via the electrodes 418 can be modified in response to a fall determination, for example with an increased sampling rate or other modification. In addition to fall detection, the accelerometer 115 (or similar sensor) can be used to determine potential body trauma due to sudden acceleration and/or deceleration (e.g., a vehicular accident, sports collision, concussion, etc.). These events could be thrombolytic, a precursor to stroke. Similar to stroke determination, these fall determinations or other movements can be employed by processing circuitry 402 when determining the seizure metric or otherwise determining whether or not the patient is experiencing, or has experienced, a seizure. For example, sensors 414 may detect head movement frequency indicative of a seizure and initiate or increase the sensing frequency of electrical signal sensing and seizure metric generation. In one example, IMD 106 may utilize the signal generated by the accelerometer, at least in part, classify a seizure as a motor onset or non-motor onset seizure. For motor onset seizures, IMD 106 may further utilize the accelerometer signal to classify a motor onset seizure as a tonic-clonic seizure or some other motor seizure type.

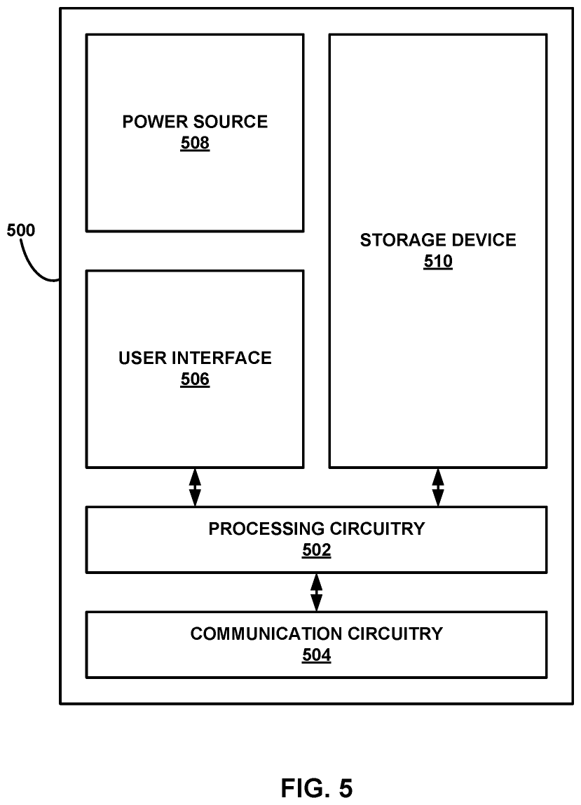

FIG. 5 is a block diagram of an example external device 500 configured to communicate with any IMD (e.g., IMD 106 or IMD 400) or sensor device described herein. External device 500 is an example of external device 108 of FIG. 1A. In the example of FIG. 5, external device 500 includes processing circuitry 502, communication circuitry 504, storage device 510, user interface 506, and power source 508.

Processing circuitry 502, in one example, may include one or more processors that are configured to implement functionality and/or process instructions for execution within external device 500. For example, processing circuitry 502 may be capable of processing instructions stored in storage device 510. Processing circuitry 502 may include, for example, microprocessors, GPUs, TPUs, DSPs, ASICs, FPGAs, or equivalent discrete or integrated logic circuitry, or a combination of any of the foregoing devices or circuitry. Accordingly, processing circuitry 502 may include any suitable structure, whether in hardware, software, firmware, or any combination thereof, to perform the functions ascribed herein to processing circuitry 502.

Communication circuitry 504 may include any suitable hardware, firmware, software or any combination thereof for communicating with another device, such as IMD 400. Under the control of processing circuitry 502, communication circuitry 504 may receive downlink telemetry from, as well as send uplink telemetry to, IMD 400, or another device. In other examples, communication circuitry 504 may also employ TCC for communicating with other devices.

Storage device 510 may be configured to store information within external device 500 during operation. Storage device 510 may include a computer-readable storage medium or computer-readable storage device. In some examples, storage device 510 includes one or more of a short-term memory or a long-term memory. Storage device 510 may include, for example, RAM, dynamic random access memories (DRAM), static random access memories (SRAM), magnetic discs, optical discs, flash memories, or forms of electrically programmable memories (EPROM) or EEPROM. In some examples, storage device 510 is used to store data indicative of instructions for execution by processing circuitry 502. Storage device 510 may be used by software or applications running on external device 500 to temporarily store information during program execution.

Data exchanged between external device 500 and IMD 400 may include operational parameters. External device 500 may transmit data including computer readable instructions which, when implemented by IMD 400, may control IMD 400 to change one or more operational parameters and/or export collected data. For example, processing circuitry 502 may transmit an instruction to IMD 400 which requests IMD 400 to export collected data (e.g., data corresponding to one or more of the physiological information, seizure metrics, stroke metrics, or accelerometer signal) to external device 500. In turn, external device 500 may receive the collected data from IMD 400 and store the collected data in storage device 510. Additionally, or alternatively, processing circuitry 502 may export instructions to IMD 400 requesting IMD 400 to update electrode combinations for stimulation or sensing.

A user, such as a clinician or patient 102, may interact with external device 500 through user interface 506. User interface 506 includes a display (not shown), such as an LCD or LED display or other type of screen, with which processing circuitry 502 may present information related to IMD 400 (e.g., stroke and/or seizure metrics). In addition, user interface 506 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 502 of external device 500 and provide input. In other examples, user interface 506 also includes audio circuitry for providing audible notifications, instructions or other sounds to patient 102, receiving voice commands from patient 102, or both. Storage device 510 may include instructions for operating user interface 506 and for managing power source 508.

Power source 508 is configured to deliver operating power to the components of external device 500. Power source 508 may include a battery and a power generation circuit to produce the operating power. In some examples, the battery is rechargeable to allow extended operation. Recharging may be accomplished by electrically coupling power source 508 to a cradle or plug that is connected to an alternating current (AC) outlet. In addition, recharging may be accomplished through proximal inductive interaction between an external charger and an inductive charging coil within external device 500. In other examples, traditional batteries (e.g., nickel cadmium or lithium ion batteries) may be used. In addition, external device 500 may be directly coupled to an alternating current outlet to operate.

In some examples, external device 500 may provide an alert to the patient or another entity (e.g., a call center) based on a stroke or seizure indication provided by IMD 400. In some examples, user interface 506 may provide an interface for presenting an alert of the detection, prediction, or classification of the condition, e.g., stroke, and for a user, e.g., the patient, a caregiver, or a clinician, to provide input overriding the detection, prediction, or classification. In this manner, systems as described herein may avoid unnecessary emergency activity resulting from a false detection by the system. Additionally, or alternatively, external device 500 may output user prompts which can be synchronized with data collection via IMD 400. For example, external device 500 may instruct the user to lift an arm, make a facial expression, etc., and IMD 400 may record physiological data while the user performs the requested actions. Moreover, external device 500 may itself analyze the patient (e.g., the patient's activity or condition in response to such prompts), for example using a camera to detect facial drooping, using a microphone to detect slurred speech, or to detect any other indicia of stroke. In some embodiments, such indicia can be compared against pre-stroke inputs (e.g., a stored baseline facial image or voice-print with baseline speech recording). Similarly, external device 500 may user one or more sensors to detect patient movement or facial activity to provide data indicative of a seizure or upcoming seizure.

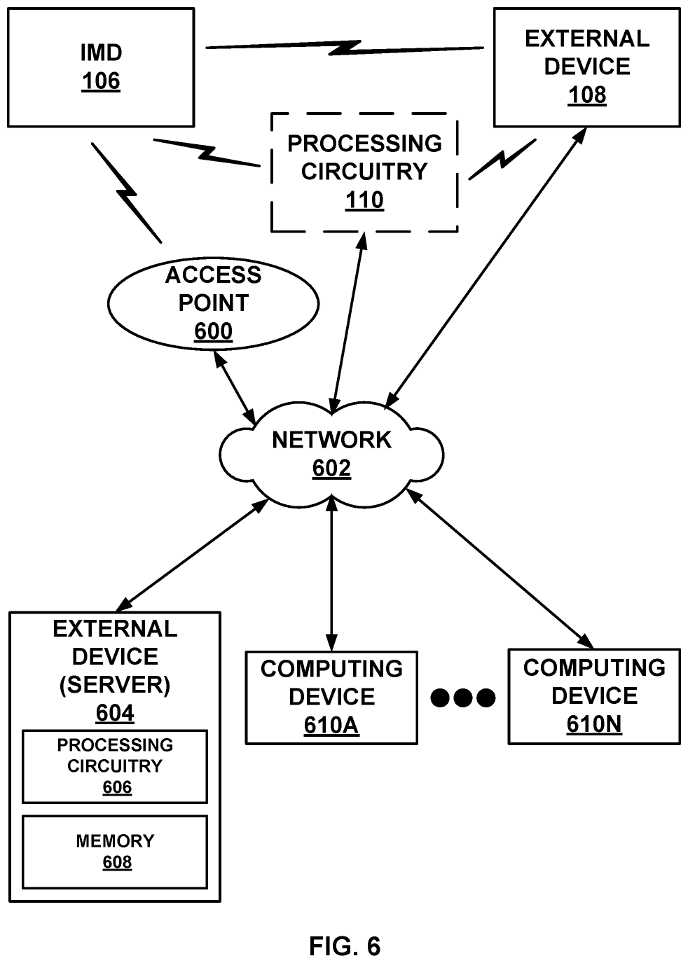

FIG. 6 is a block diagram illustrating an example system that includes an access point 600, a network 602, external computing devices, such as a server 604, and one or more other computing devices 610A-610N, which may be coupled to IMD 106, external device 108, and processing circuitry 110 via network 602, in accordance with one or more techniques described herein. In this example, IMD 106 may use communication circuitry to communicate with external device 108 via a first wireless connection, and to communication with an access point 600 via a second wireless connection. In the example of FIG. 6, access point 600, external device 108, server 604, and computing devices 610A-610N are interconnected and may communicate with each other through network 602.

Access point 600 may include a device that connects to network 602 via any of a variety of wired or wireless network connections, such as telephone dial-up, digital subscriber line (DSL), or cable modem connections. In other examples, access point 600 may be coupled to network 602 through different forms of connections, including wired or wireless connections. In some examples, access point 600 may be a user device, such as a tablet or smartphone, that may be co-located with the patient. As discussed above, IMD 106 may be configured to transmit data, such as any one or combination of an EGM signal, an accelerometer signal, and a tissue impedance signal to external device 108. In addition, access point 600 may interrogate IMD 106, such as periodically or in response to a command from the patient or network 602, in order to retrieve parameter values determined by processing circuitry of IMD 106, or other operational or patient data from IMD 106. Access point 600 may then communicate the retrieved data to server 604 via network 602.

In some cases, server 604 may be configured to provide a secure storage site for data that has been collected from IMD 106, and/or external device 108. In some cases, server 604 may assemble data in web pages or other documents for viewing by trained professionals, such as clinicians, via computing devices 610A-610N. One or more aspects of the illustrated system of FIG. 6 may be implemented with general network technology and functionality, which may be similar to that provided by the Medtronic CareLink® Network developed by Medtronic plc, of Dublin, Ireland.

Server 604 may include processing circuitry 606. Processing circuitry 606 may include fixed function circuitry and/or programmable processing circuitry. Processing circuitry 606 may include any one or more of a microprocessor, a GPU, a TPU, a controller, a DSP, an ASIC, an FPGA, or equivalent discrete or analog logic circuitry. In some examples, processing circuitry 606 may include multiple components, such as any combination of one or more microprocessors, one or more controllers, one or more DSPs, one or more ASICs, or one or more FPGAs, as well as other discrete or integrated logic circuitry. The functions attributed to processing circuitry 606 herein may be embodied as software, firmware, hardware or any combination thereof. In some examples, processing circuitry 606 may perform one or more techniques described herein based on an EGM signal, impedance signal, an accelerometer signal, or other sensor signals received from IMD 106, or parameter values determined based on such signals by IMD 106 and received from IMD 106, as examples. For example, processing circuitry 606 may perform one or more of the techniques described herein to identify significant changes in one or more physiological parameters resulting from an event, such changes resulting from a medical treatment.

Server 604 may include memory 608. Memory 608 includes computer-readable instructions that, when executed by processing circuitry 606, cause IMD 106 and processing circuitry 606 to perform various functions attributed to IMD 106 and processing circuitry 606 herein. Memory 608 may include any volatile, non-volatile, magnetic, optical, or electrical media, such as RAM, ROM, NVRAM, EEPROM, flash memory, or any other digital media.

In some examples, one or more of computing devices 610A-610N (e.g., device 610A) may be a tablet or other smart device located with a clinician, by which the clinician may program, receive alerts from, and/or interrogate IMD 106. For example, the clinician may access data corresponding to any one or combination of sensed physiological signals, an accelerometer signal, seizure metrics, stroke metrics, and other types of signals collected by IMD 106, or parameter values determined by IMD 106 based on such signals, through device 610A, such as when patient 102 is in between clinician visits, to check on a status of a medical condition. In some examples, the clinician may enter instructions for a medical intervention for patient 102 into an app in device 610A, such as based on a status of a patient condition determined by IMD 106, external device 108, processing circuitry 110, or any combination thereof, or based on other patient data known to the clinician. Device 610A then may transmit the instructions for medical intervention to another of computing devices 610A-610N (e.g., device 610B) located with patient 102 or a caregiver of patient 102. For example, such instructions for medical intervention may include an instruction to change a drug dosage, timing, or selection, to schedule a visit with the clinician, or to seek medical attention. In further examples, device 610B may generate an alert to patient 102 based on a status of a medical condition of patient 102 determined by IMD 106, which may enable patient 102 proactively to seek medical attention prior to receiving instructions for a medical intervention. In this manner, patient 102 may be empowered to take action, as needed, to address his or her medical status, which may help improve clinical outcomes for patient 102.

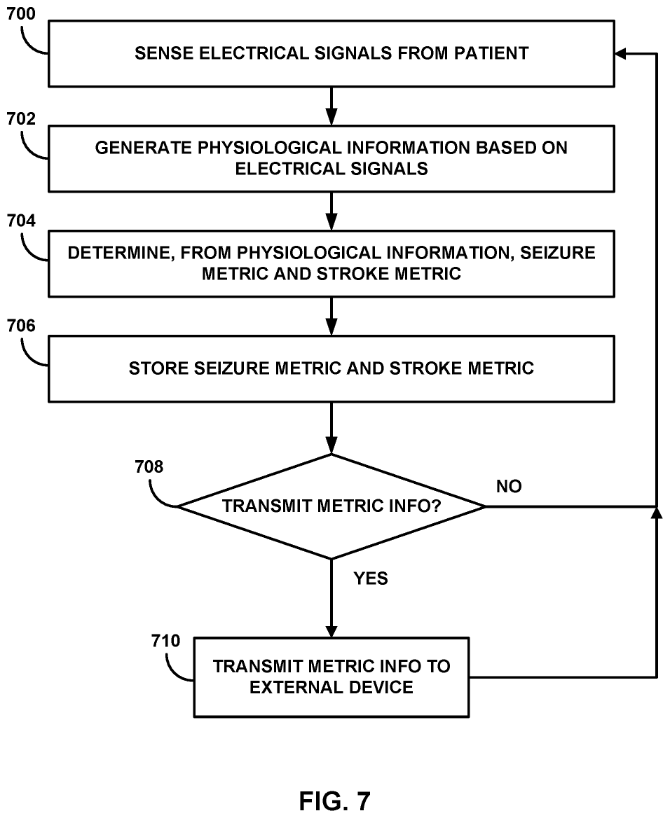

FIG. 7 is a flow diagram of an example technique for detecting at least one of a stroke or seizure of a patient. Sensing circuitry 406 and processing circuitry 402 of IMD 400 will be described as performing the techniques of example FIG. 7, but other components, devices, and systems (e.g., IMD 106 or sensor devices 210, 220, or 230) may perform similar functionality in other examples. As shown in the example of FIG. 7, sensing circuitry 406 senses electrical signals from the patient (700). Sensing circuitry 406 may sense these electrical signals from a sensing vector determined by the electrodes 418 selected for sensing. In this manner, sensing circuitry 406 may use different vectors (e.g., different electrode combinations) in order to obtain different electrical information from the patient. In some examples, sensing circuitry 406 may use different electrode combinations for detecting stroke and seizure, respectively. Sensing circuitry 406 may then generate physiological information based on the sensed electrical signals (702). Generating physiological information may include various filtering, amplification, transforms, digitization, or any other conditioning and processing that generates physiological information that can be analyzed by processing circuitry 402.

Processing circuitry 402 then receives the physiological information from sensing circuitry 406 and determines, from the physiological information, seizure metrics and stroke metrics (704). In some examples, processing circuitry 402 determines seizure metrics and stroke metrics at the same frequency and/or from the same physiological information. In other examples, processing circuitry 402 may determine seizure metrics or stroke metrics more frequently than the other. In some cases, seizures may only last a few minutes in duration, so processing circuitry 402 may determine seizure metrics more frequently than stroke metrics. Processing circuitry 706 then stores the seizure metrics and stroke metrics in memory (706). If processing circuitry 402 has instructions to transmit the metric information to an external device (such as external device 108) (“YES” branch of block 708), processing circuitry 402 may control communication circuitry to transmit the metric information to external device 108 (710). For example, processing circuitry 706 may receive a trigger to send the information such as the seizure metric or stroke metric exceeding a respective threshold that indicates a seizure or stroke is occurring or has occurred. In this manner, the external device 108 may inform the patient or a clinician that the patient may need assistance or therapeutic intervention. If processing circuitry 402 does not have instructions to transmit the metric information to an external device (such as external device 108) (“NO” branch of block 708), processing circuitry 402 continues to sense electrical signals from the patient (700).

Processing circuitry 402 may employ various techniques to determine the stroke metric and seizure metric. For example, processing circuitry 402 may generate the stroke metric using one or more different algorithms, such as using machine learning algorithms. In one example, a gradient boosting algorithm can be trained on a data set following feature extraction to generate a classifier algorithm. The classifier can be tuned by paring down features to only those related to the stroke/non-stroke condition. A sequentially backward floating feature selection approach can be employed, which sequentially removes individual features using a classifier performance metric. The classifier can then be further tuned by adjusting the frequency bins. The result of this analysis may include five features that effectively discriminate between stroke and non-stroke conditions. These example features are three frequency bins associated with the P3 electrode (5.5-7.5 Hz, 8-9.5 Hz, and 13.5-15 Hz) and two frequency bins associated with the P4 electrode (5.5-7.5 Hz and 13.5-15 Hz).

The resulting classifier may be employed by processing circuitry to make stroke/non-stroke determinations with high accuracy, such as accuracy of approximately 85%. Using this approach and the features discussed above, this example classifier may achieve relatively high accuracy while only relying on data from three electrodes: P3, P4, and the ground electrode, Pz of FIG. 1C. As such, IMD 106 or sensor device 210, for example, can generate a stroke metric that identifies stroke without requiring data from a full array of 16 or more EEG electrodes as found in conventional approaches. As described herein the stroke metric can be, for example, a binary output of stroke condition/non-stroke condition, a probabilistic indication of stroke likelihood, or other output relating to the patient's condition and likelihood of having suffered a stroke. This stroke metric can be calculated using a classifier model as described elsewhere herein.

The accuracy of any classifier can be improved by training the algorithm on larger sets of data corresponding to stroke and non-stroke EEG readings. Additionally, other physiological parameters can be added to the classifier model (e.g., fall detection as determined using an accelerometer, particular heart rhythms, gender, age, medical history, etc.). Additionally, in some examples, a classifier can be used to discriminate between ischemic and hemorrhagic strokes. Such discrimination can be particularly useful as the interventions may differ. For example, an ischemic stroke may be treated using thrombectomy, while a hemorrhagic stroke may be treated using surgery or another suitable technique.

In some examples for circuitry specific to generating physiological information (e.g., stroke information) used by processing circuitry 402 to generate stroke metrics, sensing circuitry 406 may filter the electrical signal to remove ECG artifacts. Conventionally, EEG data has been obtained via electrodes positioned over the scalp because it is a relatively noise-free location for signal acquisition. Other anatomical locations such as back of the neck have not been used, not because EEG signal is not present, but because of the noisier environment and band-overlap with other physiologic signals such as ECG. However, processing circuitry 402 may employ machine learning/adaptive neural network techniques to improve the signal extraction capability (e.g., to filter out or reduce the contribution of ECG signals from the EEG signals). One such methodology is described in “ECG Artifact Removal of EEG signal using Adaptive Neural Network” as published in IEEE Xplore 27 May 2019, which is hereby incorporated by reference in its entirety. Similarly, electrical signals associated with muscle activity may also be filtered from the EEG sensor data to remove such artifacts.

In some examples, the classification algorithm for determining that stroke metric can be, for example, an algorithm adapted from the use of artificial intelligence (e.g., machine learning, neural networks, etc.) as applied to patient stroke data to determine what type of stroke was detected. Based on the classification algorithm, in block stroke determination is made, which may be binary or probabilistic. If a stroke is detected (e.g., a probabilistic determination exceeds some pre-determined threshold, for example 85% likelihood of stroke), then processing circuitry 402 may apply an etiology classifier. In some examples, such an etiology classifier can make a determination (probabilistic or definitive) of the origin of the stroke (e.g., ischemic or hemorrhagic). Such determinations can be made based on collected EEG sensor data alone or in conjunction with additional physiological parameters or patient data. For example, the etiology classifier may determine a location of the stroke. For example, the location determination can include a left-versus-right hemisphere determination (e.g., a binary output or probabilistic result). In one example of hemisphere-specific signals, one or more electrodes may be disposed near and configured to detect brain activity data corresponding to activity at a T3 brain region and another one or more electrodes may be disposed near and configured to detect brain activity data corresponding to activity of a T4 brain region. Processing circuitry may then be configured to determine, based on the physiological information representative of hemisphere activity associated with respective electrodes at the T3 or T4 brain region, the seizure metric indicative of the seizure status of the patient and a stroke metric indicative of the stroke status of the patient. In other words, the system may utilize information regarding which hemisphere of the brain the seizure or stroke originates from as at least part of the location of the stroke, as one example. This hemisphere specific information may be obtained from locations other than T3 and T4 in other examples. In some examples, the location determination can include a more precise mapping of brain regions with particular probabilities assigned, for example a 70% probability of the stroke location being at a particular point on the patient's brain. The stroke location may be output along a spherical surface map or other suitable coordinate system for identifying the location in the patient's brain. Processing circuitry 402 may output the result of these classifiers in the form of a value of the patient metric or other value indicative of the detected type of stroke.

Processing circuitry 402 may detect seizures by generating a seizure metric that can be a binary output of seizure condition/non-seizure condition, a probabilistic indication of stroke likelihood, a type of seizure that was experienced, or other output relating to the patient's condition and likelihood of having experienced a seizure. This seizure metric can be calculated by comparing one or more features extracted from physiological information to respective thresholds, look-up tables, equations, or by applying one or more classifiers to the physiological information to employ machine learning models to the data. Machine learning models may be trained on data sensed from IMD 106 or sensor device 210, for example, in the specific patient and/or other patients that may or may not experience seizures.

In one example, IMD 400 and processing circuitry 402 may generate a seizure metric based on the following technique. Processing circuitry 402 may control sensing circuitry 406 to sense an electrical signal via an electrode combination of electrodes 418 and generate physiological information that is analyzed. Processing circuitry 402 may perform a fast Fourier transform (FFT) and determine the spectral power over a frequency band of interest (e.g., a frequency band from about 5 Hz to about 45 Hz) at approximately 2 Hz using one second of buffered electrical signal data. In some examples, the overlap of buffered data may be about 50% or about 0.5 seconds. This results in a one second average power for that particular frequency band, i.e., a short-term average of the power. In other examples, processing circuitry 402 may analyze different (e.g., smaller or larger) frequency bands, use different sampling frequencies, or different buffer sizes. Processing circuitry 402 may average the one second average power over a period of time, such as 30 minutes as one example. This averaging of the one second average power may be averaged using a cascade filter, such as a three stage cascade average filter to form a long-term average, or background, signal. An example three stage cascade average filter may include a first stage mean filter, a second stage median filter, and a third state mean filter. Processing circuitry 402 may generate a ratio of the short-term average to the long-term average to identify the changes in the brain state of the patient related to seizure.

Processing circuitry 402 may then apply the ratio of the short-term average to the long-term average to one or more thresholds to identify whether or not a seizure has occurred. In one example, processing circuitry 402 compares the ratio to dual thresholds (e.g., using a dual threshold detector) to determine if a seizure has occurred and the duration of the seizure. For example, if the ratio increases above the onset threshold, processing circuitry 402 determines that the seizure has started. Processing circuitry 402 may calculate the time until the ratio then falls below a term threshold. The onset threshold and the term threshold may be set to the same value in some examples, but in other examples, the onset and term thresholds may be different. For example, the onset threshold may be higher than the term threshold to prevent premature detection of seizure and ensure that the duration of the seizure captures the entire event. In some examples, processing circuitry 402 may also employ an onset count threshold and/or term count threshold before determining that the onset threshold or term threshold has been exceeded. Both the onset count threshold and term count threshold may establish a number of consecutive times that the ratio must exceed the onset threshold or exceed the term threshold, respectively, before processing circuitry 402 can determine that the onset threshold or term threshold has been exceeded. In this manner, brief periods of time shorter than the onset count threshold that the ratio exceeds the onset threshold will not be identified as the start of a seizure. Likewise, brief periods of time shorter than the term count threshold that the ratio drops below the onset threshold will not be identified as the end of a seizure. The seizure metric may include a value that indicates whether or a seizure was detected and/or the duration of the detected seizure using the above technique.

Processing circuitry 402 may also perform additional processing or analysis for determining the seizure metric using IMD 400 disposed on the back of the neck or head at which increased noise may affect one or more sensing vectors. For example, processing circuitry 402 (or sensing circuitry 406) may average electrical signals sensed (or average the spectral powers calculated from sensed electrical signals) from two or more different electrode combinations. Processing circuitry 402 can then apply the average electrical signals or spectral powers to the technique described above that includes an onset threshold. This process may enhance common mode rejection of any artifacts in the sensed electrical signals. Processing circuitry 402 may provide vector summation to use any linear combination (e.g., a general weighted average) of any two physical signals to form a virtual signal. In another example, processing circuitry 402 may include one or more additional median filters and/or mean filters in the cascade averaging filter of the technique described above.

In other examples, processing circuitry 402 may apply one or more ratios derived from respective electrode combinations (i.e., sensing vectors) to a decision tree seizure classifier. For example, processing circuitry 402 may determine ratios for multiple frequency bands from each available sensing vector (e.g., three vectors in the example of IMD 400 or sensor device 210) and then feed those ratios into an ensemble of decision tree seizure classifiers. In one example, the decision tree seizure classifiers may be trained off-line using gradient boosting (e.g., gradient boosting similar as discussed above with respect to the stroke metric determination). Processing circuitry 402 may employ any of these techniques to generate seizure metrics indicative of whether or not the patient has experienced a seizure. In this manner, processing circuitry 402 may leverage many different sensed signals available to IMD 400 which may reduce the impact of extraneous noise or unwanted signals on calculations in order to extract relevant features associated with brain activity.

When processing circuitry 402 transmits the stroke metric and/or seizure metric to an external device, the external device may be associated with emergency services in some examples. In some examples, the external device may include global position system (GPS) capability or other location detection technology (e.g., WiFi triangulation) such that the external device can identify, store, and/or communicate the geographic location at which the stroke metric or seizure metric occurred. The external device may then transmit the location information and/or seizure metric and/or stroke metric to another device or system via cell phone tower, satellite, or other technology. The other system may be an emergency service such as 911 or other medical service. If the technique of FIG. 7 is performed in an ambulance, for example, a device carried by the ambulance or technician may receive the metric and output information or instructions to an emergency medical technician (EMT) or other personnel in the rear of the ambulance and/or to the ambulance driver. In some embodiments, the display to the ambulance driver can include navigational information such as a map and instructions to take the patient to a particular hospital or facility with a stroke center or seizure expertise.

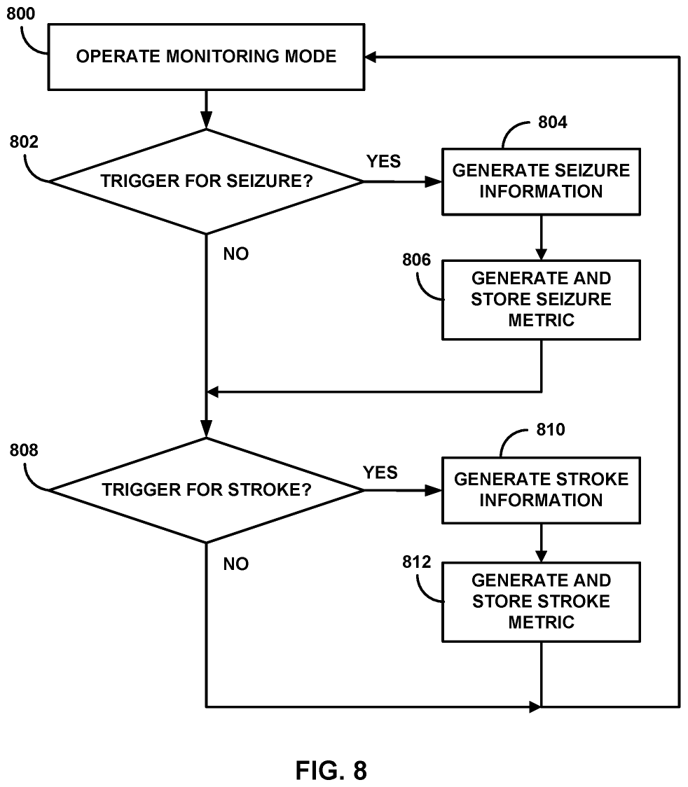

FIG. 8 is a flow diagram of an example technique for timing detection of stroke or seizure based on one or more trigger events. Sensing circuitry 406 and processing circuitry 402 of IMD 400 will be described as performing the techniques of example FIG. 8, but other components, devices, and systems (e.g., IMD 106 or sensor devices 210, 220, or 230) may perform similar functionality in other examples. As shown in the example of FIG. 8, processing circuitry 402 may operate in monitoring mode for both seizure and stroke (800). If processing circuitry 402 receives a trigger to detect seizure (“YES” branch of block 802), processing circuitry 402 controls sensing circuitry 406 to sense electrical signals and generate seizure information (which may be a part of physiological information) (804). Using the seizure information, processing circuitry 402 then generates and stores the seizure metric according to any of the example techniques described herein (806). The trigger may be a trigger event that is based on a time of day, seizure detection frequency (e.g., a timer that triggers determination at the expiration of the timer), sensed activity or movement of the patient, stroke determination, or any other information that indicates the system should perform analysis for potential seizure.

If processing circuitry 402 has not received a trigger to detect seizure (“NO” branch of block 802), processing circuitry determines whether a stroke trigger event has been received (808). If processing circuitry 402 has received the trigger to detect stroke (“YES” branch of block 808), processing circuitry 402 controls sensing circuitry 406 to sense electrical signals and generate stroke information (which may be a part of physiological information) (810). Using the stroke information, processing circuitry 402 then generates and stores the stroke metric according to any of the example techniques described herein (812). The trigger may be a trigger event that is based on a time of day, stroke detection frequency (e.g., a timer that triggers determination at the expiration of the timer), sensed activity or movement of the patient associated with stroke, seizure determination, or any other information that indicates the system should perform analysis for potential stroke. Processing circuitry 402 then continues to operate in monitoring mode (800).

In other examples, processing circuitry 402 may determine whether or not the trigger for seizure or the trigger for stroke has occurred in parallel with each other or in a different order (e.g., look for stroke triggers before seizure triggers) in the control loop. It is noted that processing circuitry 402 may calculate stroke metrics and seizure metrics in any order or simultaneously with each other.

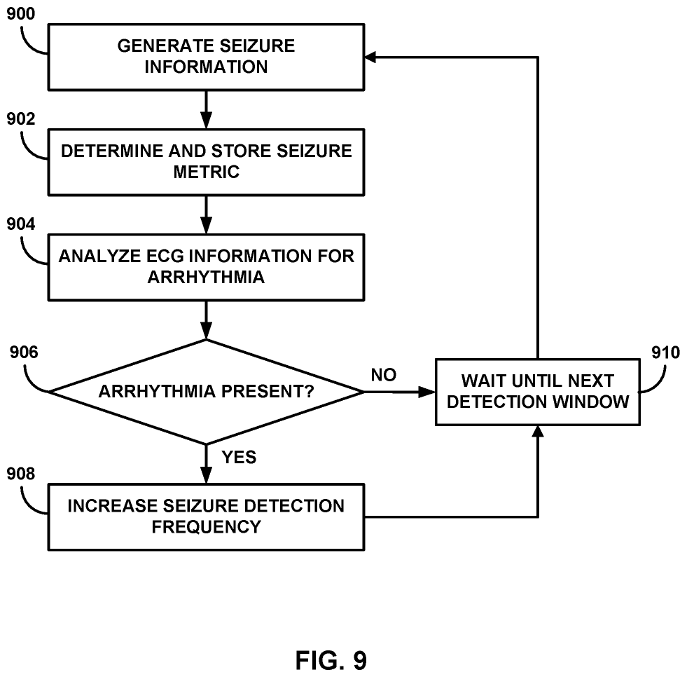

FIG. 9 is a flow diagram of an example technique for adjusting a seizure detection frequency based on arrhythmia detection. Sensing circuitry 406 and processing circuitry 402 of IMD 400 will be described as performing the techniques of example FIG. 9, but other components, devices, and systems (e.g., IMD 106 or sensor devices 210, 220, or 230) may perform similar functionality in other examples. As shown in the example of FIG. 9, processing circuitry 402 may control sensing circuitry 406 to sense electrical signals and generate seizure information (900). Processing circuitry 402 then determines, from the seizure information, a seizure metric indicative of whether or not a seizure has occurred and store the seizure metric (902).

Processing circuitry 402 then analyzes ECG information for any arrhythmia of the heart of the patient (904). In some examples, processing circuitry 402 may extract features of heart functionality (e.g., heart rate or heart rate variability) from the same physiological information generated for the purpose of calculating the seizure metric or stroke metric. In some examples, processing circuitry 402 may determine R-R intervals (the time between adjacent R-waves in the ECG) and then determine additional information from the R-R intervals, such as heart rate variability, marginal intervals or number of ectopic beats, presence of tachycardiac, a trend increasing or decreasing in the R-R intervals, or other features. In other examples, various filters may be employed to remove ECG features from the sensed signals. In this case, processing circuitry 402 may control sensing circuitry 406 to sense electrical signals and employ specific conditioning or processing to retain frequencies related to heart functionality and include that information as part of the seizure information in order to identify arrhythmias.

If processing circuitry 402 determines that an arrhythmia is present in the patient (“YES” branch of block 906), processing circuitry 402 increases the seizure detection frequency that controls how often or when processing circuitry 402 identifies whether or not a seizure is present (908). The increase in seizure detection frequency may cause the next detection window to occur earlier in time than would have occurred otherwise. If processing circuitry 402 determines that an arrhythmia is not present in the patient (“NO” branch of block 906), processing circuitry 402 waits until the next detection window (910) before generating additional seizure information (900) and determining the next seizure metric (902). In some examples, the lack of any arrhythmia present in the patient may also trigger processing circuitry 402 to set the seizure detection frequency back to a baseline or normal value in case the seizure detection frequency was previously increased due to a detected arrhythmia. Processing circuitry 402 may wait a predetermined time after the arrhythmia occurred, or ended, before setting the seizure detection frequency back to the baseline or normal value.

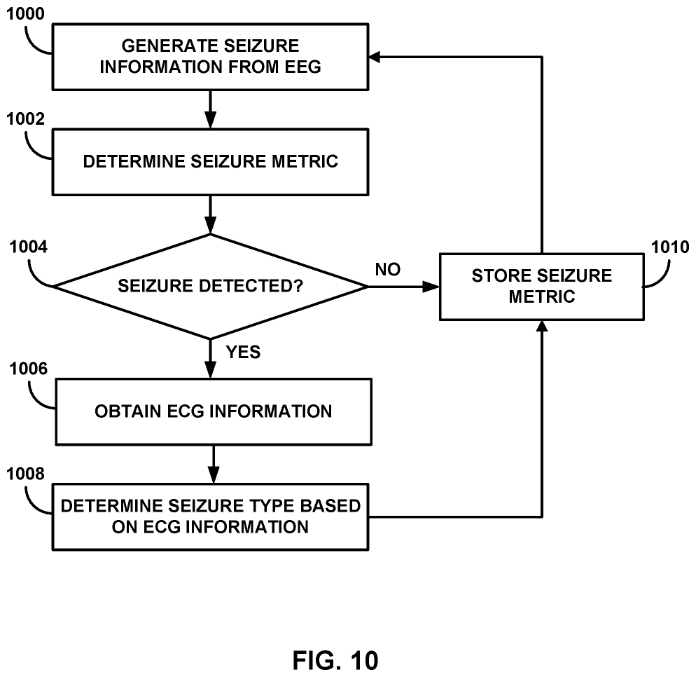

FIG. 10 is a flow diagram of an example technique for determining a seizure type based on electrocardiogram information. Sensing circuitry 406 and processing circuitry 402 of IMD 400 will be described as performing the techniques of example FIG. 10, but other components, devices, and systems (e.g., IMD 106 or sensor devices 210, 220, or 230) may perform similar functionality in other examples. As shown in the example of FIG. 10, processing circuitry 402 may control sensing circuitry 406 to sense electrical signals and generate seizure information from a sensed EEG (1000). Processing circuitry 402 then determines, from the seizure information, a seizure metric indicative of whether or not a seizure has occurred (1002).

If the seizure metric is indicative that no seizure has occurred (“NO” branch of block 1004), processing circuitry 402 stores the seizure metric (1010) and continues to generate seizure information as programmed (1000). If the seizure metric is indicative that a seizure has occurred (“YES” branch of block 1004), processing circuitry 402 obtains ECG information (1006). As described in FIG. 9, processing circuitry 402 may control sensing circuitry 406 to sense an electrical signal between one or more vectors and then condition or process the sensed signal to at least retain ECG information. In some examples, sensing circuitry 406 and/or processing circuitry 402 may filter sensed signals in order to extract cardiac features such as R-waves or P-waves in order to determine R-R intervals and/or heart rate information that may be included in the ECG information. The ECG information may thus be indicative of heart function. Processing circuitry 402 may then determine the seizure type based on one or more features of the ECG information (1008). For example, processing circuitry 402 may classify the seizure as an epileptic seizure in response to determining decreased heart rate variability or other changes in heart rate variability during the seizure. In other examples, processing circuitry 402 may determine that the seizure is a tonic-clonic seizure in response to determining that the patient had an increase in heart rate during the seizure. In some cases, processing circuitry 402 may differentiate between epileptic seizures and other types of seizures, such as syncope and psychogenic events. After classifying the seizure, processing circuitry 402 stores the seizure metric and seizure classification (1010) before continuing to monitor the patient (1000). In some examples, processing circuitry 402 may also initiate communication with an external device in response to identifying that a seizure, or a particular type of seizure, has occurred.

The following examples are described herein.

### Example 1

A system includes a memory; a plurality of electrodes; sensing circuitry configured to: sense, via at least two electrodes of the plurality of electrodes, electrical signals from a patient; and generate, based on the electrical signals, physiological information; processing circuitry configured to: receive, from the sensing circuitry, the physiological information; determine, based on the physiological information, a seizure metric indicative of a seizure status of the patient and a stroke metric indicative of a stroke status of the patient; and store the seizure metric and the stroke metric in the memory; and a housing carrying the plurality of electrodes and containing both of the sensing circuitry and the processing circuitry.

### Example 2

The system of example 1, wherein the physiological data comprises brain activity data.

### Example 3

The system of any of examples 1 or 2, wherein the plurality of electrodes are configured to detect brain activity data corresponding to activity in at least one of a P3, Pz, or P4 brain region.

### Example 4

The system of any of examples 1 through 3, wherein the housing is configured to be disposed at or adjacent to a rear portion of a neck or skull of the patient.

### Example 5

The system of any of examples 1 through 4, wherein the plurality of electrodes are configured to detect brain activity data corresponding to activity in at least one of a T3 or T4 brain region.

### Example 6

The system of example 5, wherein the processing circuitry is configured to determine, based on the physiological information representative of hemisphere activity associated with respective electrodes at the T3 or T4 brain region, the seizure metric indicative of the seizure status of the patient and a stroke metric indicative of the stroke status of the patient.

### Example 7

The system of any of examples 1 through 6, wherein the housing is configured to be implanted within the patient.

### Example 8

The system of example 7, wherein the housing is configured to be implanted subcutaneously.

### Example 9

The system of any of examples 1 through 8, wherein the housing is configured to be disposed on an external surface of skin of the patient.

### Example 10

The system of any of examples 1 through 9, wherein the physiological data comprises electrical brain activity data and electrical heart activity data, and wherein the sensing circuitry comprises: first circuitry configured to generate the electrical brain activity from the electrical signals; and second circuitry different from the first circuitry and configured to generate the electrical heart activity data from the electrical signals.

### Example 11

The system of any of examples 1 through 10, further comprising an accelerometer within the housing, the accelerometer configured to generate motion data representative of motion of the patient, and wherein the processing circuitry is configured to determine, based on the physiological data that includes the motion data, the seizure metric and the stroke metric.

### Example 12

The system of any of examples 1 through 11, wherein the processing circuitry is configured to determine, based on the motion data, that the patient has fallen, and wherein the processing circuitry is configured to determine the stroke metric based on the determination that the patient has fallen.

### Example 13

The system of example 12, wherein the processing circuitry is configured to: determine that a characteristic of the motion data exceeds a threshold; and responsive to determining that the characteristic of the motion data exceeds the threshold, determine at least one of the seizure metric or the stroke metric.

### Example 14

The system of any of examples 1 through 13, wherein the physiological information comprises electrocardiogram information, and wherein the processing circuitry is configured to: determine, based on the seizure metric, that the patient has experienced a seizure; and select, based on the electrocardiogram information and from a plurality of seizure types, one seizure type representative of the seizure experienced by the patient.

### Example 15

The system of any of examples 1 through 14, wherein the processing circuitry is configured to determine the seizure metric at a seizure detection frequency different than a stroke detection frequency of the stroke metric.

### Example 16

The system of example 15, wherein the seizure detection frequency is greater than the stroke detection frequency.

### Example 17

The system of any of examples 1 through 16, wherein the processing circuitry is configured to: determine, based on an electrocardiogram signal of the physiological information, an arrhythmia of a heart of the patient; responsive to determining the arrhythmia, increase a seizure detection frequency that controls determination of the seizure metric; and determine the seizure metric according to the seizure detection frequency.

### Example 18

The system of any of examples 1 through 17, further includes determine a geographic location of the patient; and transmit the geographic location and at least one of the seizure metric or the stroke metric to an emergency service.

### Example 19

A method includes sensing, by sensing circuitry and via at least two electrodes of a plurality of electrodes, electrical signals from a patient; generating, by the sensing circuitry and based on the electrical signals, physiological information receiving, by processing circuitry and from the sensing circuitry, the physiological information; determining, by the processing circuitry and based on the physiological information, a seizure metric indicative of a seizure status of the patient and a stroke metric indicative of a stroke status of the patient; and storing, by the processing circuitry, the seizure metric and the stroke metric in the memory, wherein a housing carries the plurality of electrodes and contains both of the sensing circuitry and the processing circuitry.

### Example 20

The method of example 19, wherein the physiological data comprises brain activity data.

### Example 21

The method of any of examples 19 and 20, wherein the plurality of electrodes are configured to detect brain activity data corresponding to activity in at least one of a P3, Pz, or P4 brain region.

### Example 22

The method of any of examples 19 through 21, wherein the housing is configured to be disposed at or adjacent to a rear portion of a neck or skull of the patient.

### Example 23

The method of any of examples 19 through 22, wherein the plurality of electrodes are configured to detect brain activity data corresponding to activity in at least one of a T3 or T4 brain region.

### Example 24

The method of example 23, wherein determining the seizure metric indicative of the seizure status of the patient and the stroke metric indicative of the stroke status of the patient comprises determining, based on the physiological information representative of hemisphere activity associated with respective electrodes at the T3 or T4 brain region, the seizure metric indicative of the seizure status of the patient and a stroke metric indicative of the stroke status of the patient.

### Example 25

The method of any of examples 19 through 24, wherein the housing is configured to be implanted within the patient.

### Example 26

The method of example 25, wherein the housing is configured to be implanted subcutaneously.

### Example 27

The method of any of examples 19 through 26, wherein the housing is configured to be disposed on an external surface of skin of the patient.

### Example 28

The method of any of examples 19 through 27, wherein the physiological data comprises electrical brain activity data and electrical heart activity data, and wherein the method further comprises: generating, by first circuitry of the sensing circuitry, the electrical brain activity from the electrical signals; and generating, by second circuitry different from the first circuitry, the electrical heart activity data from the electrical signals.

### Example 29

The method of any of examples 19 through 28, further comprising generating, by an accelerometer within the housing, motion data representative of motion of the patient, and wherein determining the seizure metric and the stroke metric comprises determining, based on the physiological data that includes the motion data, the seizure metric and the stroke metric.

### Example 30

The method of example 29, further comprising determining, based on the motion data, that the patient has fallen, and wherein determining the stroke metric comprises determining the stroke metric based on the determination that the patient has fallen.

### Example 31

The method of example 30, wherein determining the stroke metric comprises determining the stroke metric based on the determination that the patient has fallen and a time window representative of the time duration from when the patient was determined to have fallen.

### Example 32

The method of any of examples 29 through 31, further includes determining that a characteristic of the motion data exceeds a threshold; and responsive to determining that the characteristic of the motion data exceeds the threshold, determining at least one of the seizure metric or the stroke metric.

### Example 33

The method of any of examples 19 through 32, wherein the physiological information comprises electrocardiogram information, and wherein the method further comprises: determining, based on the seizure metric, that the patient has experienced a seizure; and selecting, based on the electrocardiogram information and from a plurality of seizure types, one seizure type representative of the seizure experienced by the patient.

### Example 34

The method of any of examples 19 through 33, wherein determining the seizure metric comprises determining the seizure metric at a seizure detection frequency different than a stroke detection frequency of the stroke metric.

### Example 35

The method of example 34, wherein the seizure detection frequency is greater than the stroke detection frequency.

### Example 36

The method of any of examples 19 through 35, further includes determining, based on an electrocardiogram signal of the physiological information, an arrhythmia of a heart of the patient; and responsive to determining the arrhythmia, increase a seizure detection frequency that controls determination of the seizure metric, wherein determining the seizure metric comprises determining the seizure metric according to the seizure detection frequency.

### Example 37

The method of any of examples 19 through 36, further includes controlling telemetry circuitry to transmit at least one of the seizure metric or the stroke metric to an external device; determining, by the external device, a geographic location of the patient; and transmitting, by the external device, the geographic location and at least one of the seizure metric or the stroke metric to an emergency service.

### Example 38

A computer-readable medium includes control sensing circuitry to sense, via at least two electrodes of a plurality of electrodes, electrical signals from a patient; control the sensing circuitry to generate, based on the electrical signals, physiological information; receive, from the sensing circuitry, the physiological information; determine, based on the physiological information, a seizure metric indicative of a seizure status of the patient and a stroke metric indicative of a stroke status of the patient; and store the seizure metric and the stroke metric in the memory, wherein a housing carries the plurality of electrodes and contains both of the sensing circuitry and the processing circuitry.

The techniques described in this disclosure may be implemented, at least in part, in hardware, software, firmware, or any combination thereof. For example, various aspects of the techniques may be implemented within one or more microprocessors, DSPs, ASICs, FPGAs, GPUs, TPUs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. A system comprising:
a memory;
a plurality of electrodes;
sensing circuitry configured to:
sense, via at least two electrodes of the plurality of electrodes, electrical signals from a patient; and
generate, based on the electrical signals, physiological information;
processing circuitry configured to:
receive, from the sensing circuitry, the physiological information;
determine, based on the physiological information, a seizure metric indicative of a seizure status of the patient and a stroke metric indicative of a stroke status of the patient; and
store the seizure metric and the stroke metric in the memory; and
a housing carrying the plurality of electrodes and containing both of the sensing circuitry and the processing circuitry.

2. The system of claim 1, wherein the physiological data comprises brain activity data.

3. The system of claim 1, wherein the plurality of electrodes are configured to detect brain activity data corresponding to activity in at least one of a P3, Pz, or P4 brain region.

4. The system of claim 1, wherein the housing is configured to be disposed at or adjacent to a rear portion of a neck or skull of the patient.

5. The system of claim 1, wherein the plurality of electrodes are configured to detect brain activity data corresponding to activity in at least one of a T3 or T4 brain region.

6. The system of claim 5, wherein the processing circuitry is configured to determine, based on the physiological information representative of hemisphere activity associated with respective electrodes at the T3 or T4 brain region, the seizure metric indicative of the seizure status of the patient and a stroke metric indicative of the stroke status of the patient.

7. The system of claim 1, wherein the housing is configured to be implanted within the patient.

8. The system of claim 7, wherein the housing is configured to be implanted subcutaneously.

9. The system of claim 1, wherein the physiological data comprises electrical brain activity data and electrical heart activity data, and wherein the sensing circuitry comprises:
first circuitry configured to generate the electrical brain activity from the electrical signals; and
second circuitry different from the first circuitry and configured to generate the electrical heart activity data from the electrical signals.

10. The system of claim 1, further comprising an accelerometer within the housing, the accelerometer configured to generate motion data representative of motion of the patient, and wherein the processing circuitry is configured to determine, based on the physiological data that includes the motion data, the seizure metric and the stroke metric.

11. The system of claim 1, wherein the processing circuitry is configured to determine, based on the motion data, that the patient has fallen, and wherein the processing circuitry is configured to determine the stroke metric based on the determination that the patient has fallen.

12. The system of claim 1, wherein the physiological information comprises electrocardiogram information, and wherein the processing circuitry is configured to:
determine, based on the seizure metric, that the patient has experienced a seizure; and
select, based on the electrocardiogram information and from a plurality of seizure types, one seizure type representative of the seizure experienced by the patient.

13. The system of claim 1, wherein the processing circuitry is configured to determine the seizure metric at a seizure detection frequency different than a stroke detection frequency of the stroke metric, and wherein the seizure detection frequency is greater than the stroke detection frequency.

14. The system of claim 1, wherein the processing circuitry is configured to:
determine, based on an electrocardiogram signal of the physiological information, an arrhythmia of a heart of the patient;
responsive to determining the arrhythmia, increase a seizure detection frequency that controls determination of the seizure metric; and
determine the seizure metric according to the seizure detection frequency.

15. The system of claim 1, further comprising telemetry circuitry within the housing and an external device, wherein the processing circuitry is configured to control the telemetry circuitry to transmit at least one of the seizure metric or the stroke metric to the external device, and wherein the external device is configured to:
determine a geographic location of the patient; and
transmit the geographic location and at least one of the seizure metric or the stroke metric to an emergency service.

16. A method comprising:
sensing, by sensing circuitry and via at least two electrodes of a plurality of electrodes, electrical signals from a patient;
generating, by the sensing circuitry and based on the electrical signals, physiological information;
receiving, by processing circuitry and from the sensing circuitry, the physiological information;
determining, by the processing circuitry and based on the physiological information, a seizure metric indicative of a seizure status of the patient and a stroke metric indicative of a stroke status of the patient; and
storing, by the processing circuitry, the seizure metric and the stroke metric in the memory, wherein a housing carries the plurality of electrodes and contains both of the sensing circuitry and the processing circuitry.

17. The method of claim 16, wherein the plurality of electrodes are configured to detect brain activity data corresponding to activity in at least one of a P3, Pz, or P4 brain region.

18. The method of claim 16, wherein the physiological data comprises electrical brain activity data and electrical heart activity data, and wherein the method further comprises:
generating, by first circuitry of the sensing circuitry, the electrical brain activity from the electrical signals; and
generating, by second circuitry different from the first circuitry, the electrical heart activity data from the electrical signals.

19. The method of claim 16, further comprising generating, by an accelerometer within the housing, motion data representative of motion of the patient, and wherein determining the seizure metric and the stroke metric comprises determining, based on the physiological data that includes the motion data, the seizure metric and the stroke metric.

20. The method of claim 19, further comprising determining, based on the motion data, that the patient has fallen, and wherein determining the stroke metric comprises determining the stroke metric based on the determination that the patient has fallen.

21. The method of claim 16, wherein the physiological information comprises electrocardiogram information, and wherein the method further comprises:
determining, based on the seizure metric, that the patient has experienced a seizure; and
selecting, based on the electrocardiogram information and from a plurality of seizure types, one seizure type representative of the seizure experienced by the patient.

22. A computer-readable medium comprising instructions that, when executed, cause processing circuitry to:
control sensing circuitry to sense, via at least two electrodes of a plurality of electrodes, electrical signals from a patient;
control the sensing circuitry to generate, based on the electrical signals, physiological information;
receive, from the sensing circuitry, the physiological information;
determine, based on the physiological information, a seizure metric indicative of a seizure status of the patient and a stroke metric indicative of a stroke status of the patient; and
store the seizure metric and the stroke metric in the memory, wherein a housing carries the plurality of electrodes and contains both of the sensing circuitry and the processing circuitry.
